SEQUENCE LISTING (11 sequences)

SEQ ID NO: 1 - protein, 539 aa, Human respirovirus 3
  source 1..539: note = GenBank AGW51052.1
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIIILIMMIILFIINITIITIAIKYYRIQKRNRVDQNDKPYVLTNK

SEQ ID NO: 2 - protein, 539 aa, Human respirovirus 3
  source 1..539: note = SWISS-PROT: P06828.2
MPTSILLIITTMIMASFCQIDITKLQHVGVLVNSPKGMKISQNFETRYLILSLIPKIEDSNSCGDQQIKQYKRLLDRLIIPLYDGLRLQKDVIVSNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYRVDSISYNIQNREWYIPLPSHIMTKGAFLGGADVKECIEAFSSYICPSDPGFVLNHEMESCLSGNISQCPRTVVKSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWHQSSTTIIIVLIMIIILFIINVTIIIIAVKYYRIQKRNRVDQNDKPYVLTNK

SEQ ID NO: 3 - protein, 580 aa, Human respirovirus 1
  source 1..580: note = GenBank BAS30410.1
MQSSEVLLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNKESTQIIIIIIVCVLIIIICSILYYLYRVRRLLIMINSTNNSPINAYTLESRMKNPYMGNHSNKIRSSILHTYNNQIYPQLLSDVVRK

SEQ ID NO: 4 - protein, 551 aa, Human orthorubulavirus 2
  source 1..551: note = GenBank AAA46842.1
MHHLHPMIVCIFVMYTGIVGSDAIAGDQLLNIGVIQSKIRSLMYYTDGGASFIVVKLLPNLPPSNGTCNITSLDAYNVTLFKLLTPLIENLSKISTVTDTKTRQKRFAGVVVGLAALGVATAAQITAAVAIVKANANAAAINNLASSIQSTNKAVSDVIDASRTIATAVQAIQDRINGAIVNGITSASCRAHDALIGSILNLYLTELTTIFHNQITNPALTPLSIQALRILLGSTLPIVIESKLNTNFNTAELLSSGLLTGQIISISPMYMQMLIQINVPTFIMQPGAKVIDLIAISANHKLQEVVVQVPNRILEYANELQNYPANDCVVTPNSVFCRYNEGSPIPESQYQCLRGNLNSCTFTPIIGNFLKRFAFANGVLYANCKSLLCRCADPPHVVSQDDTQGISIIDIKRCSEMMLDTFSFRITSTFNATYVTDFSMINANIVHLSPLDLSNQINSINKSLKSAEDWIADSNFFANQARTAKTLYSLSAIALILSVITLVVVGLLIAYIIKLVSQIHQFRSLAATTMFHRENPAFFSKNNHGNIYGIS

SEQ ID NO: 5 - protein, 544 aa, Human orthorubulavirus 4
  source 1..544: note = GenBank AGU90035.1
MGVKGSSLIMIGLLISPITNLDITHLMNLGTVPTAIRSLVYYTYTKPSYLTVDLIPNLKNLDQNCNYSSLNYYNKTALSLIQPIADNINRLTKPITSSEVQSRFFGAVIGTIALGVATAAQVTAAIGLAKAQENAKLILTLKKAATETNEAVRDLANSNKIVVKMISAIQNQINTIIQPAIDQINCQIKDLQVANILNLYLTEITTVFHNQLTNPALESISIQALKSLLGSTLPEVLSKLDLNNISAASVMASGLIKGQIIAVDIPTMTLVLMVQIPSISPLRQAKIIDLTSITIHTNSQEVQAVVPARVLEIGSEILGFDGSVCQITKDTVFCPYNDAYVLPIQQKRCLQGQTRDCVFTPVAGTFPRRFLTTYGTIVANCRDLVCSCLRPPQIIYQPDENPVTIIDKDLCTTLTLDSITIEIQKSINSTFRREVVLESTQVRSLTPLDLSTDLNQYNQLLKSAEDHIQRSTDYLNSINPSIVNNNAIIILIILCILLILTVTICIIWLKYLTNEVKNVARNQRLNRDADLFHRIPSQIPVPRQ

SEQ ID NO: 6 - protein, 538 aa, Mumps orthorubulavirus
  source 1..538: note = GenBank BAA94388.1
MKVFLVTCLGFAVFSSSVCVNINILQQIGYIKQQVRQLSYYSQSSSSYIVVKLLPNIQPTDNSCEFKSVTQYNKTLSNLLLPIAENINNIASPSSGSRRHKRFAGIAIGIAALGVATAAQVTAAVSLVQAQTNARAIAAMKNSIQATNRAVFEVKEGTQRLAIAVQAIQDHINTIMNTQLNNMSCQILDNQLATSLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVNVNSKIGAIIVAALVLSILSIIISLLFCCWAYVATKEIRRINFKTNHINTISSSVDDLIRY

SEQ ID NO: 7 - protein, 529 aa, Mammalian orthorubulavirus 5
  source 1..529: note = GenBank AAC95515.1
MGTIIQFLVVSCLLAGAGSLDPAALMQIGVIPTNVRQLMYYTEASSAFIVVKLMPTIDSPISGCNITSISSYNATVTKLLQPIGENLETIRNQLIPTRRRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFLTRFVLFDGIVYANCRSMLCKCMQPAAVILQPSSSPVTVIDMYKCVSLQLDNLRFTITQLANVTYNSTIKLESSQILSIDPLDISQNLAAVNKSLSDALQHLAQSDTYLSAITSATTTSVLSIIAICLGSLGLILIILLSVVVWKLLTIVVANRNRMENFVYHK

SEQ ID NO: 8 - protein, 562 aa, synthetic construct
  UNSURE 141: note = X is K or R
  SIGNAL 1..18
  REGION 19..481: note = Human respirovirus 3 F protein ectodomain
  REGION 482..513: note = GCN4 CC tri2 trimerization domain
  REGION 515..522: note = HRVC3 site
  REGION 524..531: note = octahistidine tag
  REGION 534..541: note = strep tag
  REGION 542..552: note = linker
  REGION 555..562: note = strep tag
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAXSDIEKLKEAIRDTNKAVQSVQSSVGNQIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPSLEVLFQGPGHHHHHHHHSAWSHPQFEKGGGSGGGGSGGSAWSHPQFEK

SEQ ID NO: 9 - protein, 595 aa, synthetic construct
  SIGNAL 1..18
  UNSURE 141: note = X is K or R
  REGION 19..481: note = Human respirovirus 3 F protein ectodomain
  REGION 482..513: note = GCN4 CC tri trimerization domain
  REGION 514..517: note = linker
  REGION 546..547: note = foldon trimerization motif of T4 fibritin
  REGION 546..547: note = linker
  REGION 548..555: note = HRVC3 site
  REGION 557..564: note = octahistidine tag
  REGION 567..574: note = strep tag
  REGION 575..585: note = linker
  REGION 588..595: note = strep tag
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAXSDIEKLKEAIRDTNKAVQSVQSSVGNQIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPSGGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 10 - protein, 562 aa, synthetic construct
  UNSURE 141: note = X is K or R
  SIGNAL 1..18
  REGION 19..481: note = Human respirovirus 3 F protein ectodomain
  REGION 482..513: note = GCN4 CC tri2 trimerization domain
  REGION 515..522: note = HRVC3 site
  REGION 524..531: note = octahistidine tag
  REGION 534..541: note = strep tag
  REGION 542..552: note = linker
  REGION 555..562: note = strep tag
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAXSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPSLEVLFQGPGHHHHHHHHSAWSHPQFEKGGGSGGGGSGGSAWSHPQFEK

SEQ ID NO: 11 - protein, 595 aa, synthetic construct
  UNSURE 141: note = X is K or R
  SIGNAL 1..18
  REGION 19..481: note = Human respirovirus 3 F protein ectodomain
  REGION 482..513: note = GCN4 CC tri trimerization domain
  REGION 514..517: note = linker
  REGION 518..545: note = foldon trimerization motif of T4 fibritin
  REGION 546..547: note = linker
  REGION 548..555: note = HRVC3 site
  REGION 557..564: note = octahistidine tag
  REGION 567..574: note = strep tag
  REGION 575..585: note = linker
  REGION 588..595: note = strep tag
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAXSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPSGGSGYIPEAPRDGQAYVRKDGEWVLLSTFLGRSLEVLFQGPGHHHHHHHHSAWSHPQFEKGGGSGGGGSGGSAWSHPQFEK

(19) United States
(12) Patent Application Publication
(10) Pub. No.: US 2026/0306613 A1
(43) Pub. Date: Oct. 8, 2026

(54) PREFUSION-STABILIZED HUMAN PARAINFLUENZA VIRUS 3 F PROTEINS

(71) Applicant: BOARD OF REGENTS, THE UNIVERSITY OF TEXAS SYSTEM, Austin, TX (US)

(72) Inventors: Jason McLELLAN, Austin, TX (US); Patrick BYRNE, Austin, TX (US); Elizabeth BLADE, Austin, TX (US); Ching-Lin HSIEH, Austin, TX (US)

(73) Assignee: BOARD OF REGENTS, THE UNIVERSITY OF TEXAS SYSTEM, Austin, TX (US)

(21) Appl. No.: 19/146,737

(22) PCT Filed: Jan. 9, 2024
(86) PCT No.: PCT/US2024/010814

§ 371 Date: Jul. 9, 2025

Related U.S. Application Data
(60) Provisional application No. 63/479,127, filed on Jan. 9, 2023.

(51) Int. Cl. A61K 39/155; A61K 47/68; C07K 14/005

(52) U.S. Cl. CPC A61K 39/155; A61K 47/6811; C07K 14/005; C07K 2319/03; C07K 2319/73; C12N 2760/18622; C12N 2760/18634

(57) ABSTRACT

Provided herein are engineered parainfluenza virus fusion protein (PIV F) polypeptides. In some aspects, the engineered PIV F polypeptides exhibit enhanced conformational stability and/or antigenicity. Methods are also provided for use of the engineered PIV F polypeptides as diagnostics, in screening platforms, and/or in vaccine compositions.

### REFERENCE TO RELATED APPLICATIONS

The present application claims the priority benefit of U.S. provisional application No. 63/479,127, filed Jan. 9, 2023, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Jan. 5, 2024, is named UTFBP1325WO_ST26.xml and is 32,897 bytes in size.

### BACKGROUND

### 1. Field

The present disclosure relates generally to the fields of medicine, virology, and immunology. More particularly, it concerns engineered parainfluenza virus fusion protein (PIV F) polypeptides and uses thereof.

### 2. Description of Related Art

Parainfluenza viruses 1, 2 3 and 4 (PIVs 1-4) cause mild to severe respiratory illness in humans. As members of the Paramyxoviridae family, PIVs are enveloped, single-stranded, negative-sense RNA [ssRNA(-)] viruses. Seasonal PIV infection is associated with about 40% of pediatric hospitalizations for lower respiratory tract infection, along with about 75% of diagnosed cases of croup. Infection with PIVs can occur throughout life. Most adults are at low risk for severe disease from an PIV infection, however infection in immunocompromised people and the elderly can sometimes lead to severe or life-threatening lower respiratory disease. Though development of a vaccine against PIV is of importance for public health, none has been licensed to date.

Long-term surveillance of respiratory viruses in the United States has identified PIV3 is the most common PIV serotype associated with symptomatic disease in children and adults, followed by PIVs 1, 2, and 4. The genomes of PIVs encode several envelope glycoproteins, one of which is the fusion glycoprotein (F). F glycoprotein is a fusogen that is required for virus entry into cells and an important target for neutralizing antibody (nAb) responses to infection. PIV F proteins share varying degrees of sequence conservation, ranging from 20-50% identical and 35-65% similarity among exemplary F proteins from the Respirovirus and Orthorubulavirus genera. While PIV vaccines that incorporate F subunit antigens have been under development, no PIV vaccines or antiviral therapeutics are approved for use in humans. Vaccines that provide durable protection, as well as therapies that avert mortality and morbidity, are therefore desirable.

Safe and effective immunogenic compositions and therapeutics are thus needed to protect against PIV infections and their associated sequelae. Diagnostic reagents to detect immune responses to PIV, to guide the design of F-based PIV vaccines, and to support the development of therapeutic or prophylactic antibodies against PIV are also needed.

### SUMMARY

As such, provided herein are engineered proteins having at least one amino acid substitution relative to the amino acid sequence of a native Respirovirus or Orthorubulavirus F protein (i.e., SEQ ID NOs: 1-7), wherein the engineered proteins are stabilized in the prefusion conformation of the Respirovirus or Orthorubulavirus F. The engineered proteins may specifically bind to a Respirovirus or Orthorubulavirus F protein prefusion-specific antibody.

In one embodiment, provided herein are engineered proteins comprising a Respirovirus or Orthorubulavirus fusion protein (preferably, a parainfluenza virus fusion protein (PIV F)) ectodomain having at least 90% sequence identity to amino acids 19-481 of SEQ ID NO: 1 or 2, said engineered protein comprising at least one substitution or set of substitutions selected from the group consisting of: H27C/F437C; H27C/T439C; H27C/P440C; H27C/I443C; H27Y; V28M; V30I; N33C/K295C; G37C/S337C; S41C/P283C; Y48C/I169C; Y48C/I169C/A140Y; L49C/L278C; L49F; L49W; I50C/A171C; 150C/T277C; I50W; S52C/K173C; S52C/S275C; S52L; L53C/S174C; P55C/V175C; P55C/Q176C; K56C/N155C; I57F; E58C/I183C; G64C/G196C; G64C/G200C; Q67C/L199C; Q67C/G200C; Y71C/L203C; L86C/V266C; Q89C/A131C; K90Y; I93C/G116C; V94C/G116C; T95C/G116C; T117P; I118C/G381C; A119C/G381C; L120C/G381C; A123P; T124P; S125C/P374C; S125P; S125W; A126P; I128F; I128W; L134C/I267C; A137C/I267C; I144W; L147C/AI71C; I151C/A171C; A157C/Q176C; A157C/D177C; A157F; V158L; Q159C/A171C; L168Q; V170I; V170M; A171V; K173Q; V175L; V175P; V179L; E182F; E182W; P185C/A195C; G191P; G200E; I201F; I201W; A202T; E209W; I213C/I226C; I213C/G230C; G219C/E333C; L228C/V264C; L228E; L228W; R236W; R236Y; S246V; L256Y; V264F; V264W; V266F; V266W; S275F; S275M; T277F; T277L; T277W; L278F; L278W; V280F; V280W; R281Y; L282F; L282W; D327C/P344C; A334S; G345M; F346C/T369C; F346C/S370C; N349P; L356F; S361C/T444C; Q362C/N447C; P364C/N447C; T366C/V449C; T367R; N380C/G433C; G381C/K431C; G382C/G433C; V384I; T413C/A436C; G433F; I443W; I443Y; V449C/I454C; V449C/D455C; V449C/I456C; V449C/S457C; A450F; L451P; D452P; I454F; D455K; I456W; S457C/V449C; S457C/1456C; K464C/V449C; S470C/K471C; K471A; K471L; W473A, and wherein the position are relative to SEQ ID NO: 1 or 2.

The engineered proteins may comprise or further comprise at least one set of paired cysteine substitutions selected from the group consisting of: H27C/F437C; H27C/T439C; H27C/P440C; H27C/I443C; N33C/K295C; G37C/S337C; S41C/P283C; Y48C/I169C; Y48C/I169C/A140Y; L49C/L278C; I50C/A171C; I50C/T277C; S52C/K173C; S52C/S275C; L53C/S174C; P55C/V175C; P55C/Q176C; K56C/N155C; E58C/I183C; G64C/G196C; G64C/G200C; Q67C/L199C; Q67C/G200C; Y71C/L203C; L86C/V266C; Q89C/A131C; T93C/G116C; V94C/G116C; T95C/G116C; I118C/G381C; A119C/G381C; L120C/G381C; S125C/P374C; L134C/I267C; A137C/I267C; L147C/A171C; I151C/A171C; A157C/Q176C; A157C/D177C; Q159C/A171C; P185C/A195C; 1213C/I226C; I213C/G230C; G219C/E333C; L228C/V264C; D327C/P344C; F346C/T369C; F346C/S370C; S361C/T444C; Q362C/N447C; P364C/N447C; T366C/V449C; N380C/G433C; G381C/K431C; G382C/G433C; T413C/A436C; V449C/1454C; V449C/D455C; V449C/I456C; V449C/S457C; S457C/V449C; S457C/I456C; K464C/V449C; and S470C/K471C. The engineered proteins may further comprise S186C/A195C paired cysteine substitutions. The paired cysteine substitutions preferably form a disulfide bond.

The engineered proteins may comprise or further comprise at least one cavity filling substitution or set of cavity filling substitutions selected from the group consisting of: H27Y; V28M; V30I; L49F; L49W; I50W; S52L; I57F; K90Y; A140Y; I144W; A157F; V158L; V170I; V170M; A171V; V175L; V179L; E182F; E182W; G200E; T201F; T201W; L228F; L228W; R236Y; S246V; L256Y; V264F; V264W; V266F; V266W; S275F; S275M; T277F; T277L; T277W; L278F; L278W; V280F; V280W; R281Y; L282F; L282W; G345M; L356F; V384I; G433F; I443W; I443Y; A450F; I454F; and I456W. The substitutions may form a salt bridge within the pair of substitutions or between the single substitution and a native amino acid in the protein.

The engineered proteins may comprise or further comprise at least one substitution or set of substitutions selected from the group consisting of: T117P; A123P; T124P; S125P; A126P; V175P; G191P; N349P; L451P; and D452P.

The engineered proteins may comprise or further comprise at least one substitution or set of substitutions selected from the group consisting of: S125W; I128F; 1128W; E209W; and R236W.

The engineered proteins may comprise or further comprise at least one substitution selected from the group consisting of: K173Q; A202T; A334S; T367R; D455K; K471A; K471L; and W473A.

The engineered proteins may comprise or further comprise an E at position 108.

The engineered proteins may comprise a combination of at least one engineered disulfide bond and at least one cavity filling substitution; or a combination of at least one engineered disulfide bond and at least one proline substitution; or a combination of at least one engineered disulfide bond, at least one cavity filing substitution, and at least one proline substitution.

The engineered proteins may comprise a set of substitutions selected from the group consisting of: G64C/G196C/V28M; G64C/G196C/V175L; G64C/G196C/V158L; G64C/G196C/A123P; G64C/G196C/S125P; G64C/G196C/I201W; G64C/G196C/L282F; G64C/G196C/L228W; G64C/G196C/R281Y; G64C/G196C/L282W; G64C/G196C/N349P; G64C/G196C/T367R; G64C/G196C/K471A; A137C/I267C/V28M; A137C/I267C/V175L; A137C/I267C/V158L; A137C/I267C/A123P; A137C/I267C/S125P; A137C/I267C/I201W; A137C/I267C/L282F; A137C/I267C/L228W; A137C/I267C/R281Y; A137C/I267C/L282W; A137C/I267C/N349P; A137C/I267C/T367R; A137C/I267C/K471A; L147C/A171C/V28M; L147C/A171C/V175L; L147C/A171C/V158L; L147C/A171C/A123P; L147C/A171C/S125P; L147C/A171C/I201W; L147C/A171C/L282F; L147C/A171C/L228W; L147C/A171C/R281Y; L147C/A171C/L282W; L147C/A171C/N349P; L147C/A171C/T367R; L147C/A171C/K471A; G64C/G196C/A137C/I267C/K471A; G64C/G196C/A I37C/I267C/V175L; G64C/G196C/A137C/I267C/S125P; G64C/G196C/A137C/I267C/S125P/V175L; G64C/G196C/A137C/I267C/T367R; G64C/G196C/A137C/I267C/K471A/S125P; G64C/G196C/A137C/I267C/K471A/S125P/T367R/V175L; G64C/G196C/A137C/I267C; G64C/G196C/A137C/I267C/K471A/S125P/L282F/V175L; G64C/G196C/L147C/A171C/K471A/S125P/L282F/V175L; G64C/G196C/L147C/A171C/A137C/I267C/K471A/S125P/L282F/V175L; G64C/G196C/L147C/A171C; G64C/G196C/L147C/A171C/V28M/V175L/I201W/L228W/S125P/T367R/K471A; G64C/G196C/A137C/I267C/V28M/VI75L/I201W/L228W/S125P/T367R/K471A; G64C/G196C/L147C/A171C/A137C/I267C/V28M/V175L/120I W/L228W/S125P/T367R/K4 71A; G64C/G196C/I151C/A171C/A137C/I267C; G64C/G196C/1151C/A171C/A137C/I267C/I231C/G230C; G64C/G196C/A137C/I267C/V28M/V175L/I201W/L228W/R281Y; G64C/G196C/A137C/I267C/V28M/V175L/I201W/L282F/L228W/R281Y; 1151C/A171C/V449C/S457C; L168Q/G64C/G196C/A137C/I267C/K471A; L168Q/G64C/G196C/A137C/I267C/V175L; L168Q/G64C/G196C/A137C/I267C/S125P; L168Q/G64C/G196C/A137C/I267C/S125P/V175L; L168Q/G64C/G196C/A137C/I267C/T367R; L168Q/G64C/G196C/A137C/I267C/K471A/S125P; L168Q/G64C/G196C/A137C/I267C/K471A/S125P/T367R/V175L; L168Q/G64C/G196C/A137C/I267C; 168Q/G64C/G196C/A137C/I267C/K471A/S125P/L282F/V175L; L168Q/G64C/G196C/I151C/A171C/A137C/I267C; L168Q/G64C/G196C/I151C/A171C/A137C/I267C/I231C/G230C; L168Q/G64C/G196C/A137C/I267C/V28M/V175L/I201W/L228W/R281Y; L168Q/G64C/G196C/A137C/I267C/V28M/V175L/I201W/L282F/L228W/R281Y; L168Q/I151C/A171C/V449C/S457C; L168Q/I151C/A171C/V449C/S457C/V28M/V175L/R281Y; I151C/A171C/S186C/A195C/V28M/V175L/R281Y; L168Q/I151C/A171C/L228W/L282F/R821Y/V175L; I151C/A171C/L228W/L282F/R821Y/V175L; I151C/A171C/L228W/L282F/R821Y/V175L/G200E/I57F; I151C/A171C/L228W/L282F/R821Y/V175L/G200E/I57F/V449C/S457C; I151C/A171C/G200E/I57F; and I151C/A171C/G200E/I57F/V449C/S457C.

The engineered proteins may comprise a substitution or set of substitutions selected from any one of the substitutions and sets of substitutions of Tables 1 and 2. Any substitution or set of substitutions may be further combined with the L168Q substitution.

The Respirovirus or Orthorubulavirus fusion protein (preferably, a parainfluenza virus fusion protein (PIV F)) ectodomain of the engineered proteins may be a human PIV (hPIV) F ectodomain. The human PIV F ectodomain may be a hPIV3 F ectodomain. The hPIV3 F ectodomain may comprise a sequence having at least 90%, at least 91%, at least 92%, at least 93%, at least 94%, at least 95%, at least 96%, at least 97%, at least 98%, at least 99%, or 100% sequence identity to amino acids 19-481 of SEQ ID NO: 1 or 2.

The engineered proteins may not comprise the cytoplasmic tail of PIV F.

The engineered proteins may be fused or conjugated to a trimerization domain. The trimerization domain may comprise a T4 fibritin trimerization domain, a GCN4 domain, a 4J4A domain, or a combination thereof. The trimerization domain may comprise a sequence selected from the group consisting of:

MKQIEDKIEEILSKIYHIENEIARIKKLIGEAP,
IEDKIEEILSKIYHIENEIARIKKLIGEAP,
MKQVEDKIEEILSKIYHIENEIARIKKLIGEAP,
VEDKIEEILSKIYHIENEIARIKKLIGEAP,
DTYLSAIEDKIEEILSKIYHIENEIARI,
LKQIVLRIMEIEARIAKIE,
LKQIVLRIMEIEARIAKIEGSGYIPEAPRDGQAYVRKDGEWVLLSTFLG,
LKQIVLRIMEIEARIAKIEGSEFNSLKQIVLRIMEIEARIAKIE,
LKQIVLRIMEIEARIAKIEGSLKQIVLRIMEIEARIAKIE,
and
LKQIVLRIMEIEARIAKIEGSLELIKLRIMEIEARIAKIEKDRAIL.

The engineered proteins may be fused or conjugated to a transmembrane domain. The transmembrane domain may comprise a PIV F protein transmembrane domain. The PIV F protein transmembrane domain may comprise the sequence IIIILIMMIILFIINITIITI. The transmembrane domain may not comprise a PIV F protein transmembrane domain.

The engineered proteins may comprise an N-terminal signal sequence.

The engineered proteins may exhibit improved solubility or stability, as compared to a native PIV F in a postfusion conformation. The engineered proteins may be immunogenic.

In one embodiment, provided herein are engineered Respirovirus or Orthorubulavirus fusion protein (preferably, a parainfluenza virus fusion protein (PIV F)) trimers comprising three engineered proteins disclosed herein. The trimers may be stabilized in a prefusion conformation relative to a trimer of native PIV F protein subunits. The trimers may comprise at least one engineered disulfide bond between subunits. The trimers may comprise at least one engineered disulfide bond between subunits selected from the group consisting of: I118C/G381C; A119C/G381C; L120C/G381C; S125C/P374C; G219C/E333C; F346C/T369C; F346C/S370C; and V449C/S457C.

In one embodiment, provided herein are nucleic acid molecules comprising a nucleotide sequence that encodes an amino acid sequence of an engineered protein disclosed herein. The nucleic acid molecules may further comprise a DNA expression vector. The nucleic acid molecules may be mRNAs. The nucleic acid molecules may be self-replicated RNA molecules. The nucleic acid molecules may comprise at least one chemical modification. The at least one chemical modification may be selected from the group consisting of pseudouridine, N1-methylpseudouridine, N1-ethylpseudouridine, N1-ethylpseudouridine, 2-thiouridine, 4′-thiouridine, 5-methylcytosine, 2-thio-1-methyl-1-deaza-pseudouri dine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In one embodiment, provided herein are pharmaceutical compositions comprising (i) an engineered protein disclosed herein, (ii) an engineered trimer disclosed herein, or (iii) a nucleic acid molecule disclosed herein; and a pharmaceutically acceptable carrier. The pharmaceutical composition may further comprise an adjuvant. The pharmaceutical composition may further comprise a further PIV antigen. The pharmaceutical composition may be formulated within a cationic lipid nanoparticle. The pharmaceutical composition may be for use in the treatment or prevention of a parainfluenza virus (PIV) infection or a disease associated with PIV infection in a subject or for use in eliciting an immune response against parainfluenza virus (PIV). The subject may be a mammal, such as a human.

In one embodiment, provided herein are methods of preventing parainfluenza virus (PIV) infection or a disease associated with PIV infection in a subject or eliciting an immune response in a subject or reducing PIV viral shedding in a subject, comprising administering to the subject an effective amount of a pharmaceutical composition provided herein. The subject may be a mammal, such as a human.

In one embodiment, provided herein are uses of (i) an engineered protein disclosed herein, (ii) an engineered trimer disclosed herein, or (iii) a nucleic acid molecule disclosed herein; or (iv) a pharmaceutical composition provided herein, in the manufacture of a medicament for the treatment or prevention of a parainfluenza virus (PIV) infection or a disease associated with PIV infection.

In one embodiment, provided herein are compositions comprising (i) an engineered protein provided herein or (ii) an engineered trimer provided herein, bound to an antibody. The antibody may specifically bind to a PIV F ectodomain in the prefusion conformation.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Sequence identity and similarity matrices for representative respiroviruses and orthorubulaviruses. A multiple sequence alignment (Clustal Omega) was analyzed to determine pairwise sequence identity and similarity for a representative set of F proteins from the Respirovirus and Orthorubulavirus genera. Points in each matrix are colored by their percent value from 0% to 100% [scale at right]. Similarity and identity were scored by the SIAS server hosted by the Immunomedicine Group at the Universidad Complutense de Madrid, SECRETARIA GENERAL DE CIENCIA, TECNOLOGIA E INNOVACION OF SPAIN [http://imed.med.ucm.es/Tools/sias.html].

FIG. 2. Clustal sequence alignment for the fusion proteins of human respiroviruses 1 and 3, also known as HPIV1 and HPIV3. The sequence identity in the soluble ectodomain is 44% and the sequence similarity is >60%. Identical residues at homologous positions are colored with white lettering on a black background; amino acids that are similar are colored with black lettering on a gray background. Non-similar residues at homologous sites are colored black with white background.

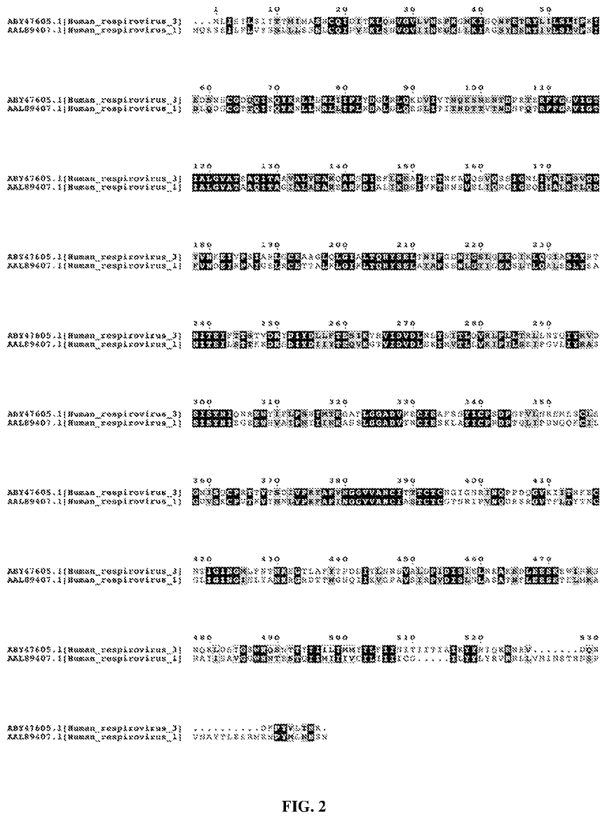

FIG. 3. Cryo-EM structure of PIV3 F base (L168Q) with a C-terminal GCN4 tag in the postfusion conformation. (Top left panel) Representative cryo-electron micrograph. (Top right panel) Representative 2D class averages. (Bottom left panel) Gold Standard Fourier Shell Correlation plot, showing the GSFSC as a function of resolution in Angstroms. Resolution values correspond to a FSC cutoff of 0.143. (Bottom right panel) Coulomb potential map, or Coulomb potential map, of PIV3 F base trimer in the postfusion conformation.

FIG. 4. SDS-PAGE of single substitutions. PIV3 F protein, purified by affinity chromatography analyzed by reducing SDS-PAGE [Coomassie stain]. Variant IDs are indicated at the top of each lane. Integrated band intensities were quantified using either ImageJ/Fiji or a Licor Odyssey CLx. Molecular weight markers are included at the left of each gel. For the bottom-most panel, both the affinity chromatography flow through [FT] and elution [E] are shown.

FIG. 5. Biolayer interferometry quantification of binding of HPIV3 F variants to the prefusion-specific antibody PIA174 IgG. Each panel shows the response (nm) as a function of time (seconds) for binding of PIV3 F variants to AHC tips. The AHC tips were functionalized with PIA174 IgG, which recognizes prefusion PIV3 F.

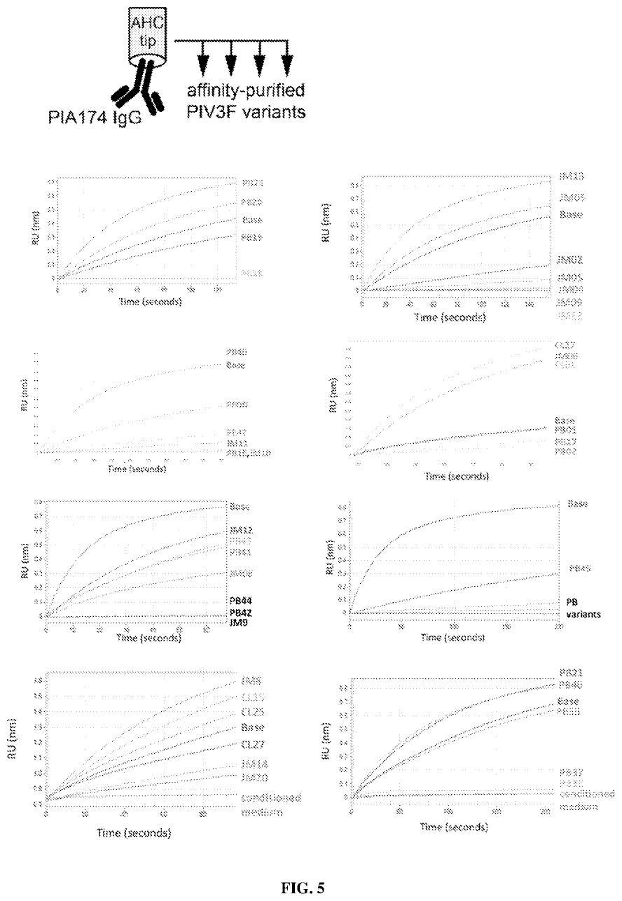

FIG. 6. Negative stain electron microscopy of the disulfide variants JM-17 (150C/A171C) and JM-20 (A137C/II267C) in prefusion conformations. The top portion of each panel provides representative 2D class averages from negative stain EM images of JM-17 [left] and JM-20 [right]. The box size is indicated below the 2D classes and is equal to 230 Angstroms. The bottom portion of each panel provides 3D reconstructions of JM-17 [left] and JM-20 [right] in the prefusion conformation.

FIG. 7. Biolayer interferometry quantification of relative expression yields of HPIV3 F variants. Each graph shows the response (nm) as a function of time (seconds) for binding of PIV3 F variants to AHC tips. The AHC tips were functionalized with MF5 IgG, which recognizes the foldon tag. Binding curves were fit to a straight line over the intial portion of the curve, which corresponded a 60 second window after dipping into media containing each variant from a small scale HEK293F expression culture.

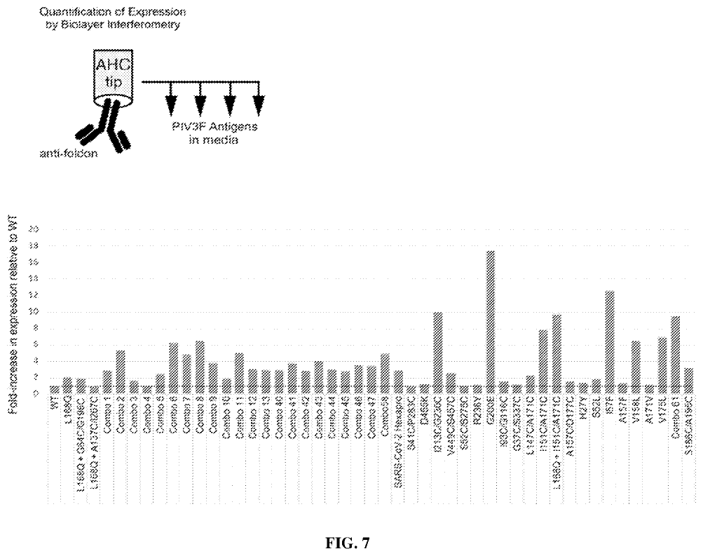

FIG. 8. SDS-PAGE quantification of combinatorial variants containing a tandem GCN4/Foldon tag on the C-terminus. Individual variant IDs and combinatorial variant IDs are indicated above each lane. Integrated band intensities are plotted below each gel, quantified in a Licor Odyssey CLx.

FIG. 9. Size-exclusion chromatography of PIV3 F variants Each graph shows the mAU as a function of elution volume for sets of PIV3 F variants. Every sample shown in this figure was transfected and purified in parallel under identical conditions. Briefly, PIV3F variants were purified by Streptactin Sepharose, then concentrated and flash frozen in liquid nitrogen. Samples were thawed and run one after another on the same Superose 6 size-exclusion chromatography (SEC) column. The UV trace for the base construct sample, L168Q, is reproduced on each graph for ease of comparison.

FIG. 10. Additional SDS-PAGE quantification of combinatorial variants containing a tandem GCN4/Foldon tag on the C-terminus. Integrated band intensities are plotted for a group of individual and combinatorial variants gel, quantified in a Licor Odyssey CLx.

FIG. 11. SDS PAGE after size-exclusion chromatography of PIV3F combinatorial variants 41 and 43. Large scale expression (500 mL) of two combinatorial variants, 41 and 43, purified first by streptactin affinity chromatography and then size-exclusion chromatography (Superose 6).

FIG. 12. Size-exclusion chromatography of PIV3F combinatorial variants 43, 56, 57 and 58. Superose 6 size-exclusion elution profiles for a set of combinatorial variants, with the Absorbance units at 280 nanometers [mAU] plotted as a function of elution volume in milliliters [mL]. In the inset table, expressed protein yields [mg/mL] and apparent melting temperatures (Tmₐₚₚ in ° C.) are indicated beside each variant ID. Tmₐₚₚ was determined by differential scanning fluorimetry. Combinatorial variant mutations are listed in Table 2.

FIGS. 13A-13B. Size-exclusion chromatography and cryo-EM of the PIV F variant L168Q+I151C/A171C. (FIG. 13A) SEC of three variants: L168Q, L168Q+I151C/A171C, and L186Q+1213C/G230C. Traces show characteristic trimer and dimer-of-trimer peaks. (FIG. 13B) Cryo-EM of the L168Q+I151C/A171C variant. Panels show 2D class averages, a 3.3 Å 3D reconstruction, and zoomed views of the model in the Coulomb potential map.

FIG. 14. Cryo-EM structure of PIV3 F base (L168Q) with a tandem GCN4/Foldon tag on the C-terminus in the prefusion conformation, without any prefusion-specific antibodies. (Top left panel) Representative cryo-electron micrograph. (Top right panel) Representative 2D class averages. (Bottom left panel) Gold Standard Fourier Shell Correlation plot, showing the GSFSC as a function of resolution in Angstroms. Resolution values correspond to a FSC cutoff of 0.143. (Bottom right panel) Coulomb potential map, or Coulomb potential map, of PIV3 F base with a C-terminal tandem GCN4/foldon tag in the trimeric prefusion conformation. PIV3 F protomers are colored blue, red, and green. The extended portion of the structure stabilized by the C-terminal tag is colored gray.

FIG. 15. Cryo-EM structure of PIV3 F combinatorial variant 43 (Combo 43) in the prefusion conformation, in complex with the prefusion-specific antibody PIA174. (Top left panel) Representative cryo-electron micrograph. (Top right panel) Representative 2D class averages. (Middle left panel) Gold Standard Fourier Shell Correlation plot, showing the GSFSC as a function of resolution in Angstroms. Resolution values correspond to a FSC cutoff of 0.143. (Middle right panel) Coulomb potential map, or Coulomb potential map, of PIV3 F variant Combo 43 (I151C/A171C) with a C-terminal tandem GCN4/foldon tag in the trimeric prefusion conformation. PIV3 F Combo 43 protomers are colored blue, red, and green. The extended portion of the structure stabilized by the C-terminal tag is colored gray. (Bottom panels) Zoomed view with all atom models of exemplary substitutions within the Coulomb potential map, shown in gray. Atoms are colored according to substitution type: disulfide, proline and cavity filling.

FIG. 16. Cryo-EM of the PIV variant I93C/G116C. Panels show a representative micrograph, 2D class averages, and a 6 Å reconstruction of the PIV F variant I93C/G116C, using the wildtype PIV F as a background (Leu at position 168).

FIG. 17. Biochemical and structural characterization of PIV3 F variant PB-68, V449C/S457C, which forms an interprotomer disulfide bond. (Top left panel) Non-reducing SDS-PAGE analysis of PIV3 variant PB-68, V449C/S457C, which runs as a disulfide-linked trimer. A control PIV3 variant that does not form an interprotomer disulfide bond, and which runs as a single protomer, was included. (Top right panel) Representative 2D class averages of PIV3 variant PB-68 from negative stain electron microscopy. (Bottom right panel) 3D negative stain EM reconstruction of PB-68. (Bottom left panel) Zoomed view of the modeled site of substitution, near the heptad repeat.

FIGS. 18A-18B. Sample stability test of PIV F variant PB-68, V449C/S457C. (FIG. 18A) Purified V449C/S457C protein was incubated at 4° C. for 0, 7 and 30 days, then analyzed by non-reducing SDS-PAGE. (FIG. 18B) A separate preparation of V449C/S457C protein was incubated at 37° C. for 1, 7, and 14 days, then analyzed by reducing and non-reducing SDS-PAGE. The band indicated with an arrow indicates the presence of non-reduced trimer. A small fraction of the sample runs at the size expected for a protomer under non-reducing conditions (low molecular weight band, near the 71 kDa MW marker), which may also form intraprotomer disulfide bonds.

FIGS. 19A-19B. Four cryo-EM structures of PIV F Combo variants 41 and 58, each in two oligomeric states. Representative class averages and Coulomb potential maps for trimers and dimer-of-trimers of (FIG. 19A) Combo variant 41 and (FIG. 19B) Combo variant 58. The resolutions of each structure are indicated beside the maps.

FIGS. 20A-20B. Variants adopt a mixture of closed and open conformations at the PIA174 binding site, which can be biased to closed by substitutions at the central trimeric interface. (FIG. 20A) Combo41 Coulomb potential maps for four unique conformations, varying from open to closed, indicative of conformational heterogeneity in the central apical region (black arrows). (FIG. 20B) Combo58 Coulomb potential map (3.4 Å) in the closed conformation. No classes were observed in an open conformation, despite extensive effort to classify based on the conformational heterogeneity in the central apical region (arrows). Atomic model and zoomed view of Combo58, with a substitution position 201.

FIG. 21. SDS-PAGE of wildtype, single and combination variants of PIV3 F. Wildtype PIV3 F and several variants (L168Q, I151C/A171C, Combo61, Combo61-0-1, Combo62-0, and Combo62-0-1) were analyzed by reducing and non-reducing SDS-PAGE. Variants containing the V449C/S457C substitution (Combo61-0-2 and Combo62-0-1) both show expected interprotomer disulfide bond trimer bands.

FIG. 22. Size-exclusion chromatograph of wildtype, single and combination variants of PIV3 F. Each graph shows the mAU as a function of elution volume for sets of PIV3 F variants. Every sample shown in this figure was transfected and purified in parallel under identical conditions. Briefly, PIV3F variants were purified by Streptactin Sepharose, then concentrated and flash frozen in liquid nitrogen. Samples were thawed and run one after another on the same Superose 6 column. The UV trace for the wildtype PIV3 F sample is reproduced in each panel for ease of comparison.

FIG. 23. Analysis of thermal stability of wildtype, single and combination variants of PIV3 F. Purified PIV F proteins, purified in parallel under identical conditions, were analyzed by differential scanning fluorimetry. Plots show the change in fluorescence with respect to temperature on the y-axis, and the temperature on the x-axis. Variants are plotted in groups, with the wildtype DSF trace reproduced in each panel for ease of comparison.

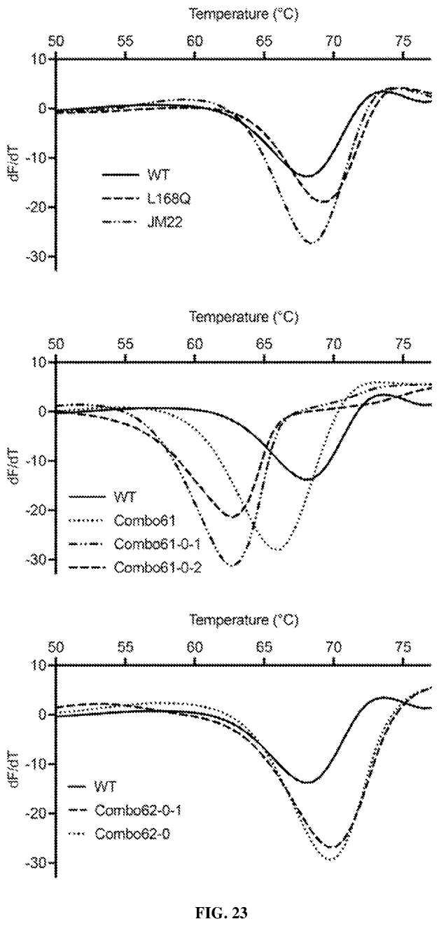

### DETAILED DESCRIPTION

Provided herein are engineered parainfluenza virus (PIV) fusion (F) proteins that have one or more amino acid substitution that stabilizes the PIV F protein in the prefusion conformation. Prefusion PIV F can be used as a vaccine antigen or reagent to detect and/or isolate antibodies in sera. The prefusion PIV F proteins described herein, and the nucleic acids that encode the proteins, may be used, for example, as potential immunogens in an immunogenic composition or a vaccine against PIV, in a method of inducing an immune response in a subject, and as diagnostic tools, among other uses.

### I. NATIVE HPIV F

An envelope glycoprotein of hPIV1, hPIV2, hPIV3, or hPIV4 that facilitates fusion of viral and cellular membranes. In nature, the F protein from hPIV1, hPIV2, hPIV3, and hPIV4 is initially synthesized as a single polypeptide precursor approximately 550 amino acids in length, designated F0. F0 includes an N-terminal signal peptide that directs localization to the endoplasmic reticulum, where the signal peptide is proteolytically cleaved. The remaining F0 residues oligomerize to form a trimer and may be proteolytically processed by a cellular protease to generate two disulfide-linked fragments, F1 and F2. In hPIV1 F the cleavage site is located approximately between residues 112/113, in hPIV2 F the cleavage site is located approximately between residues 106/107, in hPIV3 F the cleavage site is located approximately between residues 109/110, and in hPIV4 F the cleavage site is located approximately between residues 103/104. The smaller of these fragments, F2, originates from the N-terminal portion of the F0 precursor (hPIV1, approximately residues 22-113; hPIV2, approximately residues 22-106; hPIV3, approximately residues 19-109; hPIV4, approximately residues 21-103). The larger of these fragments, F1, includes the C-terminal portion of the F0 precursor (hPIV1, approximately residues 114-555; hPIV2, approximately residues 107-551; hPIV3, approximately residues 110-539; hPIV4, approximately residues 104-544) including an extracellular/lumenal region (hPIV1, approximately residues 114-497; hPIV2, approximately residues 107-493; hPIV3, approximately residues 110-493; hPIV4, approximately residues 104-486), a transmembrane domain (hPIV1, approximately residues 498-518; hPIV2, approximately residues 494-514; hPIV3, approximately residues 494-514; hPIV4, approximately residues 487-507), and a cytoplasmic tail at the C-terminus. The extracellular portion of the hPIV F protein is the hPIV F ectodomain, which includes the F2 protein and the F1 ectodomain.

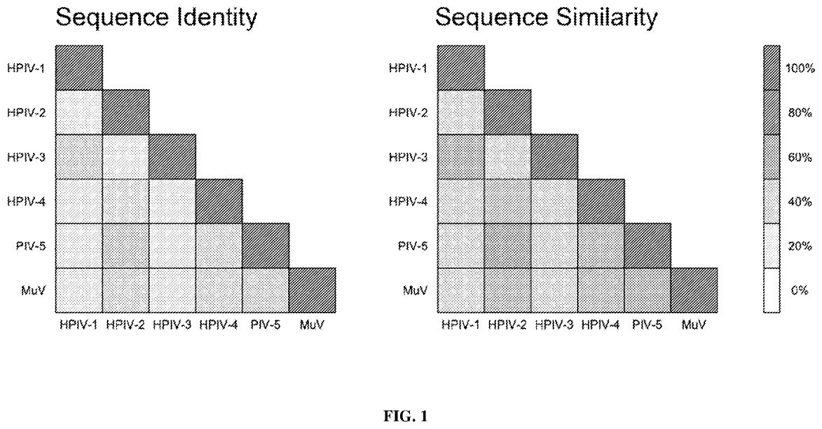

The hPIV F protein exhibits remarkable sequence conservation within hPIV subtypes, as well as other members of the Respirovirus and Orthorubulavirus genera (FIG. 1). In view of this conservation, the person of ordinary skill in the art can easily compare amino acid positions of different hPIV F proteins of the same subtype, or to the F proteins from other members of the Respirovirus and Orthorubulavirus genera. Unless context indicates otherwise, the numbering of amino acid substitutions disclosed herein is made with reference to SEQ ID NO: 1 (GenBank AGW51052.1) or 2 (SWISS-PROT: P06828.2) for hPIV3 (also known as Human respirovirus 3) F, unless otherwise indicated.

Accordingly, the term PIV F polypeptide as used herein is to be understood as the native PIV F polypeptide from any PIV strain (not limited to the human PIV3 strain), as well as any F proteins from other members of the Respirovirus and Orthorubulavirus genera. The actual residue position number may need to be adjusted for F proteins from other strains depending on the actual sequence alignment. Additional viral F proteins to which the disclosed amino acid substitutions can be applied include, for example, hPIV1 F (also known as Human respirovirus 1) (GenBank BAS30410.1; SEQ ID NO: 3), hPIV2 (also known as Human orthorubulavirus 2) F (GenBank AAA46842.1; SEQ ID NO: 4), hPIV4 (also known as Human orthorubulavirus 4) F (GenBank AGU90035.1; SEQ ID NO: 5); Mumps orthorubulavirus F (GenBank BAA94388.1; SEQ ID NO: 6); and PIV5 (also known as Mammalian orthorubulavirus 5) F (GenBank AAC95515.1; SEQ ID NO: 7).

### II. PIV F PROTEINS STABILIZED IN A PREFUSION CONFORMATION

Three PIV F protomers oligomerize in the mature F protein, which adopts a metastable prefusion conformation that is triggered to undergo a conformational change to a postfusion conformation upon contact with a target cell membrane. This conformational change exposes a hydrophobic sequence, known as the fusion peptide, which is located at the N-terminus of the F1 ectodomain, and which associates with the host cell membrane and promotes fusion of the membrane of the virus, or an infected cell, with the target cell membrane.

Provided herein are engineered PIV3 F ectodomain trimers comprising protomers comprising one or more amino acid substitutions that stabilize the F ectodomain trimer in the prefusion conformation.

As used herein, a “prefusion conformation” refers to a structural conformation adopted by the polypeptide that differs from the PIV F postfusion conformation at least in terms of molecular dimensions or three-dimensional coordinates. The prefusion conformation refers to a structural conformation adopted by PIV F prior to triggering of the fusogenic event that leads to transition of F to the postfusion conformation. Isolating PIV F in a stable prefusion conformation may be useful in informing and directing development of improved vaccines and immunogenic compositions to address the important public health problem of PIV infections. A prefusion conformation may be a conformation that can bind to a prefusion-specific antibody.

An PIV F ectodomain trimer “stabilized in a prefusion conformation” comprises one or more amino acid substitutions, deletions, or insertions compared to a corresponding native PIV F sequence that provide for increased retention of the prefusion conformation compared to PIV F ectodomain trimers formed from a corresponding native hPIV F sequence. The “stabilization” of the prefusion conformation can be, for example, energetic stabilization (for example, reducing the energy of the prefusion conformation relative to the post-fusion open conformation) and/or kinetic stabilization (for example, reducing the rate of transition from the prefusion conformation to the postfusion conformation). Additionally, stabilization of the PIV F ectodomain trimer in the prefusion conformation can include an increase in resistance to denaturation compared to a corresponding native PIV F sequence. Methods of determining if a hPIV F ectodomain trimer is in the prefusion conformation are provided herein, and include (but are not limited to) negative stain electron microscopy and antibody binding assays using a prefusion conformation specific antibody, such as the PIA3 or PIA174 antibody in the case of hPIV3.

The present disclosure provides engineered proteins that include amino acid substitutions relative to the amino acid sequence of a corresponding native hPIV3 F protein (e.g., SEQ ID NO: 1 or 2). The amino acid mutations include amino acid substitutions, deletions, or additions relative to a native hPIV3 F protein. Accordingly, the engineered proteins are mutants of native hPIV3 F proteins.

The engineered PIV F ectodomain trimer may be from a human strain of PIV other than hPIV3, such as hPIV1, hPIV2, or hPIV4. Based on the high sequence identity between hPIV3 F sequences and other hPIV F sequences, the residues of the other hPIV F sequence corresponding to those of hPIV3 F are readily attainable. Any of the amino acid substitutions (or combinations of substitutions) described herein for stabilizing hPIV3 F in its prefusion conformation can be introduced into another hPIV F sequence for prefusion stabilization.

The engineered PIV F ectodomain trimer may be from a non-human strain of PIV, such as a bovine or caprine strain of PIV. Based on the high sequence identity between human PIV F sequences and non-human PIV F sequences, the residues of the non-human PIV F sequence corresponding to those of hPIV F are readily attainable. Any of the amino acid substitutions (or combinations of substitutions) described herein for stabilizing hPIV3 F in its prefusion conformation can be introduced into a non-human PIV3 F sequence (e.g., GENBANK: AHZ90086.1 or AIW42876.1) for prefusion stabilization.

The engineered hPIV3 F ectodomain trimer comprises protomers that are “single chain” proteins wherein the F2 polypeptide and the F1 ectodomain of each protomer are directly linked or linked via a peptide linker to form a contiguous polypeptide chain. Some examples of native hPIV3 F proteins (such as GENBANK: AGW51052.1) do not include a consensus furin cleavage site between the F1 and F2 proteins; hPIV3 F immunogens based on such native hPIV3 F proteins generally do not need to be modified to produce single chain F proteins. However, other native hPIV3 F proteins (such as SWISS-PROT:P06828.2) do include a consensus furin cleavage site between the F1 and F2 proteins; hPIV3 F immunogens based on such native hPIV3 F proteins can be modified to produce single chain F proteins. Exemplary modifications include amino acid substitutions to remove the consensus furin cleavage site, such as a K108E substitution.

The protomers of the engineered PIV F ectodomain trimer include PIV F positions 19-481, and may include any of the following: an amino acid substitution (such as K108E) to remove the consensus furin cleavage site between F2 and F1 (if the consensus site is present in the native sequence), either K or R at position 87, either T or S at position 95, either K or R at position 141, either V or I at position 165, either L or Q at position 168, either K or R at position 295, either T or V at position 267, either T or K at position 369, and either D or N at position 441.

The engineered PIV F ectodomain trimers may be a soluble protein complex, for example, for use as a recombinant subunit vaccine. In several such embodiments, the protomers of the engineered PIV F ectodomain trimer can each comprise a C-terminal linkage to a trimerization domain, such as a GCN4 trimerization domain. The trimerization domain promotes trimerization and stabilization of the membrane proximal aspect of the engineered PIV F ectodomain trimer. For example, a C-terminal residue of the protomers of the engineered PIV F ectodomain trimer (such as a residue of the stem region of the trimer) can be directly linked to the trimerization domain, or indirectly linked to the trimerization domain via a peptide linker. Exemplary linkers include glycine and glycine-serine linkers. Non-limiting examples of exogenous multimerization domains that promote stable trimers of soluble recombinant proteins include: the GCN4 leucine zipper, the trimerization motif from the lung surfactant protein (Hoppe et al. 1994 FEBS Lett 344:191-195), collagen (McAlinden et al. 2003 J Biol Chem 278:42200-42207) any of which can be linked to the C-terminus of the protomers of a engineered PIV F ectodomain to promote trimerization, as long as the recombinant PIV F ectodomain trimer retains the prefusion conformation. In some examples, the protomers of the engineered PIV F ectodomain trimer can be linked to a GCN4 trimerization domain, for example, each protomer in the trimer can include a C-terminal linkage to the GCN4 trimerization domain, such as a linkage to any one of hPIV3 F positions 475-485, such as hPIV3 F position 481.

The engineered PIV F ectodomain trimer may be a membrane anchored protein complex, for example, for use in an attenuated virus or virus like particle vaccine. Membrane anchoring can be accomplished, for example, by C-terminal linkage of the protomers of the engineered PIV F ectodomain trimer to a transmembrane domain and optionally a cytoplasmic tail, such as an PIV F transmembrane domain and cytoplasmic tail. One or more peptide linkers (such as a gly-ser linker, for example, a 10 amino acid glycine-serine peptide linker) can be used to link the protomers of the engineered PIV F ectodomain trimer to the transmembrane domain. A non-limiting example of a transmembrane domain for use with the disclosed embodiments includes an hPIV3 F transmembrane domain.

The engineered proteins may possess certain beneficial characteristics, such as being immunogenic. The engineered proteins may possess increased immunogenic properties or improved stability in the prefusion conformation, as compared to the corresponding native hPIV3 F protein. Stability refers to the degree to which a transition from prefusion conformation to postfusion conformation of the hPIV3 F is hindered or prevented. The engineered proteins may display one or more introduced mutations as described herein, which may also result in improved stability in the prefusion conformation. The introduced amino acid mutations in the hPIV3 F protein include amino acid substitutions, deletions, and/or additions. The mutations in the amino acid sequences of the engineered proteins may be amino acid substitutions, insertions, and/or deletions relative to a native hPIV3 F ectodomain.

Several modes of stabilizing the engineered protein conformation include, without limitation, amino acid substitutions that introduce disulfide bonds (both intraprotomer and interprotomer), modify salt bridges, introduce electrostatic interactions, introduce hydrogen bonds, introduce prolines, fill cavities, alter the packing of residues, and combinations thereof, as compared to a native hPIV3 F protein.

The engineered proteins may be isolated, i.e., separated from hPIV3 F proteins having a postfusion conformation. Thus, the engineered proteins may be, for example, at least 80% isolated, at least 90% isolated, at least 95% isolated, at least 98% isolated, at least 99% isolated, or at least 99.9% isolated from hPIV3 F polypeptides in a postfusion conformation. The engineered proteins may specifically bind to an hPIV3 F prefusion-specific antibody.

It will be understood that a homogeneous population of engineered proteins in a particular conformation can include variations (such as polypeptide modification variations, e.g., glycosylation state), that do not alter the conformational state of the engineered proteins.

The population of engineered proteins may remain homogeneous over time. For example, the engineered proteins, when dissolved in aqueous solution, may form a population of proteins stabilized in the prefusion conformation for at least 12 hours, at least 24 hours, at least 48 hours, at least one week, at least two weeks, or more. A person of ordinary skill in the art will appreciate that the engineered proteins provided herein are useful to elicit immune responses in mammals to hPIV3.

The engineered proteins may include cysteine substitutions that are introduced, as compared to a native PIV F protein. The engineered protein may include any one of 1, 2, 3, 4, 5, 6, 7, 8, 9, or 10 cysteine substitutions. Without being bound by theory or mechanism, the cysteine substitutions described herein are believed to facilitate stability of the polypeptide in a conformation that is not the PIV F postfusion conformation. The introduced cysteine substitutions may be introduced by protein engineering, for example, by including one or more substituted cysteine residues that form a disulfide bond. The amino acid positions of the cysteines may be within a sufficiently close distance for formation of a disulfide bond in the prefusion, and not postfusion, conformation of the PIV F protein.

The engineered proteins may include a recombinant PIV F protein stabilized in a prefusion conformation by a disulfide bond between cysteines that are introduced into a pair of amino acid positions that are close to each other in the prefusion conformation and more distant in the postfusion conformation. The pair of cysteines may both be present in a single protomer, thus forming an intraprotomer disulfide bond, or the pair of cysteines may be in different protomers, thus forming an interprotomer disulfide bond.

Exemplary cysteine substitutions as compared to a native PIV F protein include any disulfide bond substitutions in Table 1, the numbering of which is based on the numbering of SEQ ID NO: 1. The engineered proteins may include a combination of two or more of the disulfide bonds between paired cysteine residues listed in Table 1.

The engineered proteins may include a combination of two or more different types of mutations selected from engineered disulfide bond mutations, cavity filling mutations, and proline mutations. The engineered proteins may include at least one disulfide bond mutation and at least proline mutation. The engineered proteins may include at least one cysteine substitution and at least one cavity filling substitution. The engineered proteins may include at least one cysteine substitution and at least one charge reduction substitution. The engineered proteins may include at least one mutation selected from any one of the mutations, or sets of mutations, in Table 1 or 2.

TABLE 1
Engineered hPIV3 F proteins (all positions
relative to SEQ ID NO: 1 or 2 or 8-11)

| Construct ID | Mutation(s)^{a} | Type |
|---|---|---|
| Base | L168Q | electrostatic interaction |
| CL-01 | V28M | cavity filling |
| CL-02 | V30I | cavity filling |
| CL-03 | V170I | cavity filling |
| CL-04 | V170M | cavity filling |
| CL-05 | S246V | cavity filling |
| CL-06 | S275M | cavity filling |
| CL-07; PB-19 | V280W | cavity filling |
| CL-08 | G345M | cavity filling |
| CL-09 | L356F | cavity filling |
| CL-10 | V384I | cavity filling |
| CL-11; LB-3 | H27C/T439C | disulfide |
| CL-12 | N33C/K295C | disulfide |
| CL-13 | K56C/N155C | disulfide |
| CL-14 | E58C/I183C | disulfide |
| CL-15 | G64C/G196C | disulfide |
| CL-16 | G64C/G200C | disulfide |
| CL-17; PB-72 | Q67C/G200C | disulfide |
| CL-18 | A157C/Q176C | disulfide |
| CL-19 | L228C/V264C | disulfide |
| CL-20 | N380C/G433C | disulfide |
| CL-21 | G381C/K431C | disulfide |
| CL-22 | T413C/A436C | disulfide |
| CL-23; PB-46; | V449C/S457C | interprotomer DS |
| PB-68 | | |
| CL-24 | A334S | polar |
| CL-25 | T367R | polar |
| CL-26 | T117P | proline |
| CL-27 | S125P | proline |
| CL-28 | A126P | proline |
| CL-29; PB-53 | P55C/V175C | disulfide |
| CL-30 | S186C/A195C | disulfide |
| CL-31 | S361C/T444C | disulfide |
| JM-01 | H27Y | cavity filling |
| JM-02 | S52L | cavity filling |
| JM-03 | I57F | cavity filling |
| JM-04 | K90Y | cavity filling |
| JM-05 | A157F | cavity filling |
| JM-06 | V158L | cavity filling |
| JM-07 | A171V | cavity filling |
| JM-08 | V175L | cavity filling |
| JM-09 | V179L | cavity filling |
| JM-10 | L256Y | cavity filling |
| JM-11 | S275F | cavity filling |
| JM-12 | T277L | cavity filling |
| JM-13 | R281Y | cavity filling |
| JM-14 | G37C/S337C | disulfide |
| JM-15 | Y48C/I169C | disulfide |
| JM-16 | Y48C/I169C/A140Y | disulfide |
| JM-17 | I50C/A171C | disulfide |
| JM-18 | S52C/K173C | disulfide |
| JM-19 | L53C/S174C | disulfide |
| JM-20 | A137C/I267C | disulfide |
| JM-21 | L147C/A171C | disulfide |
| JM-22; PB-52 | I151C/A171C | disulfide |
| JM-23 | A157C/D177C | disulfide |
| JM-24; PB-38 | Q159C/A171C | disulfide |
| JM-25 | P364C/N447C | disulfide |
| JM-26 | A202T | hydrogen bond |
| JM-27 | K173Q | polar |
| PB-01 | L49F | cavity filling |
| PB-02 | L49W | cavity filling |
| PB-03 | I50W | cavity filling |
| PB-04 | I144W | cavity filling |
| PB-05 | E182F | cavity filling |
| PB-06 | E182W | cavity filling |
| PB-07 | I201W | cavity filling |
| PB-08 | I201F | cavity filling |
| PB-09 | L228F | cavity filling |
| PB-10 | L228W | cavity filling |
| PB-11 | V264F | cavity filling |
| PB-12 | V264W | cavity filling |
| PB-13 | V266F | cavity filling |
| PB-14 | V266W | cavity filling |
| PB-15 | T277F | cavity filling |
| PB-16 | T277W | cavity filling |
| PB-17 | L278F | cavity filling |
| PB-18 | L278W | cavity filling |
| PB-20 | L282F | cavity filling |
| PB-21 | L282W | cavity filling |
| PB-22 | L134C/I267C | disulfide |
| PB-23 | P185C/A195C | disulfide |
| PB-24 | T366C/V449C | disulfide |
| PB-25 | S125W | domain interface |
| PB-26 | I128F | domain interface |
| PB-27 | I128W | domain interface |
| PB-28 | E209W | domain interface |
| PB-29 | R236W | domain interface |
| PB-30 | T124P | proline |
| PB-31 | V280F | cavity filling |
| PB-32 | A123P | proline |
| PB-33 | V175P | proline |
| PB-34 | G191P | proline |
| PB-35 | N349P | proline |
| PB-36; PB-42 | D452P | proline |
| PB-37 | Q89C/A131C | disulfide |
| PB-39 | G382C/G433C | disulfide |
| PB-40 | K471A | charge ablation |
| PB-41 | K471L | charge ablation |
| PB-43 | S457C/V449C | disulfide |
| PB-44 | V449C/D455C | disulfide |
| PB-45 | V449C/I456C | disulfide |
| PB-47 | I456W | cavity filling |
| PB-48 | I443W | cavity filling |
| PB-49 | W473A | helix propensity |
| PB-50 | S470C/K471C | disulfide |
| LB-1 | K464C/V449C | disulfide |
| LB-2 | V449C/I454C | disulfide |
| LB-4 | H27C/F437C | disulfide |
| LB-5 | L451P | proline |
| LB-6 | S457C/I456C | disulfide |
| LB-7 | A450F | cavity filling |
| LB-8 | G433F | cavity filling |
| LB-9 | I443Y | cavity filling |
| LB-10 | I454F | cavity filling |
| LB-11 | Q362C/N447C | disulfide |
| PB-51 | D327C/P344C | disulfide |
| PB-54 | P55C/Q176C | disulfide |
| PB-55 | F346C/T369C | interprotomer DS |
| PB-56 | F346C/S370C | interprotomer DS |
| PB-57 | S125C/P374C | interprotomer DS |
| PB-58 | S41C/P283C | disulfide |
| PB-59 | D455K | charge balance |
| PB-60 | L86C/V266C | disulfide |
| PB-61 | I213C/I226C | disulfide |
| PB-62 | I213C/G230C | disulfide |
| PB-63 | L49C/L278C | disulfide |
| PB-64 | Q67C/L199C | disulfide |
| PB-65 | Y71C/L203C | disulfide |
| PB-66 | H27C/P440C | disulfide |
| PB-67 | H27C/I443C | disulfide |
| PB-69 | S52C/S275C | disulfide |
| PB-70 | I50C/T277C | disulfide |
| PB-71 | G219C/E333C | interprotomer DS |
| PB-73 | R236Y | cavity |
| PB-74 | G200E | cavity |
| PB-75 | I93C/G116C | disulfide |
| PB-76 | V94C/G116C | disulfide |
| PB-77 | T95C/G116C | disulfide |
| PB-78 | I118C/G381C | interprotomer DS |
| PB-79 | A119C/G381C | interprotomer DS |
| PB-80 | L120C/G381C | interprotomer DS |

^{a}All constructs can also optionally include the L168Q substitution

TABLE 2
Exemplary combinations of substitutions (all positions
are relative to SEQ ID NO: 1 or 2 or 8-11)

| Construct ID | Mutation(s) | Type |
|---|---|---|
| Combo1 | G64C_G196C | disulfide |
| | V28M | cavity filling |
| Combo1-1 | L168Q | electrostatic interaction |
| | G64C_G196C | disulfide |
| | V28M | cavity filling |
| Combo2 | G64C_G196C | disulfide |
| | V175L | cavity filling |
| Combo2-1 | L168Q | electrostatic interaction |
| | G64C_G196C | disulfide |
| | V175L | cavity filling |
| Combo3 | G64C_G196C | disulfide |
| | V158L | cavity filling |
| Combo3-1 | L168Q | electrostatic interaction |
| | G64C_G196C | disulfide |
| | V158L | cavity filling |
| Combo4 | G64C_G196C | disulfide |
| | A123P | proline |
| Combo4-1 | L168Q | electrostatic interaction |
| | G64C_G196C | disulfide |
| | A123P | proline |
| Combo5 | G64C_G196C | disulfide |
| | S125P | proline |
| Combo5-1 | L168Q | electrostatic interaction |
| | G64C_G196C | disulfide |
| | S125P | proline |
| Combo6 | G64C_G196C | disulfide |
| | I201W | cavity filling |
| Combo6-1 | L168Q | electrostatic interaction |
| | G64C_G196C | disulfide |
| | I201W | cavity filling |
| Combo7 | G64C_G196C | disulfide |
| | L282F | cavity filling |
| Combo7-1 | L168Q | electrostatic interaction |
| | G64C_G196C | disulfide |
| | L282F | cavity filling |
| Combo8 | G64C_G196C | disulfide |
| | L228W | cavity filling |
| Combo8-1 | L168Q | electrostatic interaction |
| | G64C_G196C | disulfide |
| | L228W | cavity filling |
| Combo9 | G64C_G196C | disulfide |
| | R281Y | cavity filling |
| Combo9-1 | L168Q | electrostatic interaction |
| | G64C_G196C | disulfide |
| | R281Y | cavity filling |
| Combo10 | G64C_G196C | disulfide |
| | L282W | cavity filling |
| Combo10-1 | L168Q | electrostatic interaction |
| | G64C_G196C | disulfide |
| | L282W | cavity filling |
| Combo11 | G64C_G196C | disulfide |
| | N349P | proline |
| Combo11-1 | L168Q | electrostatic interaction |
| | G64C_G196C | disulfide |
| | N349P | proline |
| Combo12 | G64C_G196C | disulfide |
| | T367R | polar |
| Combo12-1 | L168Q | electrostatic interaction |
| | G64C_G196C | disulfide |
| | T367R | polar |
| Combo13 | G64C_G196C | disulfide |
| | K471A | charge ablation |
| Combo13-1 | L168Q | electrostatic interaction |
| | G64C_G196C | disulfide |
| | K471A | charge ablation |
| Combo14 | A137C_I267C | disulfide |
| | V28M | cavity filling |
| Combo14-1 | L168Q | electrostatic interaction |
| | A137C_I267C | disulfide |
| | V28M | cavity filling |
| Combo15 | A137C_I267C | disulfide |
| | V175L | cavity filling |
| Combo15-1 | L168Q | electrostatic interaction |
| | A137C_I267C | disulfide |
| | V175L | cavity filling |
| Combo16 | A137C_I267C | disulfide |
| | V158L | cavity filling |
| Combo16-1 | L168Q | electrostatic interaction |
| | A137C_I267C | disulfide |
| | V158L | cavity filling |
| Combo17 | A137C_I267C | disulfide |
| | A123P | proline |
| Combo17-1 | L168Q | electrostatic interaction |
| | A137C_I267C | disulfide |
| | A123P | proline |
| Combo18 | A137C_I267C | disulfide |
| | S125P | proline |
| Combo18-1 | L168Q | electrostatic interaction |
| | A137C_I267C | disulfide |
| | S125P | proline |
| Combo19 | A137C_I267C | disulfide |
| | I201W | cavity filling |
| Combo19-1 | L168Q | electrostatic interaction |
| | A137C_I267C | disulfide |
| | I201W | cavity filling |
| Combo20 | A137C_I267C | disulfide |
| | L282F | cavity filling |
| Combo20-1 | L168Q | electrostatic interaction |
| | A137C_I267C | disulfide |
| | L282F | cavity filling |
| Combo21 | A137C_I267C | disulfide |
| | L228W | cavity filling |
| Combo21-1 | L168Q | electrostatic interaction |
| | A137C_I267C | disulfide |
| | L228W | cavity filling |
| Combo22 | A137C_I267C | disulfide |
| | R281Y | cavity filling |
| Combo22-1 | L168Q | electrostatic interaction |
| | A137C_I267C | disulfide |
| | R281Y | cavity filling |
| Combo23 | A137C_I267C | disulfide |
| | L282W | cavity filling |
| Combo23-1 | L168Q | electrostatic interaction |
| | A137C_I267C | disulfide |
| | L282W | cavity filling |
| Combo24 | A137C_I267C | disulfide |
| | N349P | proline |
| Combo24-1 | L168Q | electrostatic interaction |
| | A137C_I267C | disulfide |
| | N349P | proline |
| Combo25 | A137C_I267C | disulfide |
| | T367R | polar |
| Combo25-1 | L168Q | electrostatic interaction |
| | A137C_I267C | disulfide |
| | T367R | polar |
| Combo26 | A137C_I267C | disulfide |
| | K471A | charge ablation |
| Combo26-1 | L168Q | electrostatic interaction |
| | A137C_I267C | disulfide |
| | K471A | charge ablation |
| Combo27 | L147C_A171C | disulfide |
| | V28M | cavity filling |
| Combo27-1 | L168Q | electrostatic interaction |
| | L147C_A171C | disulfide |
| | V28M | cavity filling |
| Combo28 | L147C_A171C | disulfide |
| | V175L | cavity filling |
| Combo28-1 | L168Q | electrostatic interaction |
| | L147C_A171C | disulfide |
| | V175L | cavity filling |
| Combo29 | L147C_A171C | disulfide |
| | V158L | cavity filling |
| Combo29-1 | L168Q | electrostatic interaction |
| | L147C_A171C | disulfide |
| | V158L | cavity filling |
| Combo30 | L147C_A171C | disulfide |
| | A123P | proline |
| Combo30-1 | L168Q | electrostatic interaction |
| | L147C_A171C | disulfide |
| | A123P | proline |
| Combo31 | L147C_A171C | disulfide |
| | S125P | proline |
| Combo31-1 | L168Q | electrostatic interaction |
| | L147C_A171C | disulfide |
| | S125P | proline |
| Combo32 | L147C_A171C | disulfide |
| | I201W | cavity filling |
| Combo32-1 | L168Q | electrostatic interaction |
| | L147C_A171C | disulfide |
| | I201W | cavity filling |
| Combo33 | L147C_A171C | disulfide |
| | L282F | cavity filling |
| Combo33-1 | L168Q | electrostatic interaction |
| | L147C_A171C | disulfide |
| | L282F | cavity filling |
| Combo34 | L147C_A171C | disulfide |
| | L228W | cavity filling |
| Combo34-1 | L168Q | electrostatic interaction |
| | L147C_A171C | disulfide |
| | L228W | cavity filling |
| Combo35 | L147C_A171C | disulfide |
| | R281Y | cavity filling |
| Combo35-1 | L168Q | electrostatic interaction |
| | L147C_A171C | disulfide |
| | R281Y | cavity filling |
| Combo36 | L147C_A171C | disulfide |
| | L282W | cavity filling |
| Combo36-1 | L168Q | electrostatic interaction |
| | L147C_A171C | disulfide |
| | L282W | cavity filling |
| Combo37 | L147C_A171C | disulfide |
| | N349P | proline |
| Combo37-1 | L168Q | electrostatic interaction |
| | L147C_A171C | disulfide |
| | N349P | proline |
| Combo38 | L147C_A171C | disulfide |
| | T367R | polar |
| Combo38-1 | L168Q | electrostatic interaction |
| | L147C_A171C | disulfide |
| | T367R | polar |
| Combo39 | L147C_A171C | disulfide |
| | K471A | charge ablation |
| Combo39-1 | L168Q | electrostatic interaction |
| | L147C_A171C | disulfide |
| | K471A | charge ablation |
| Combo40 | L168Q | electrostatic interaction |
| | G64C_G196C | disulfide |
| | A137C_I267C | disulfide |
| | K471A | charge ablation |
| Combo40-0 | G64C_G196C | disulfide |
| | A137C_I267C | disulfide |
| | K471A | charge ablation |
| Combo41 | L168Q | electrostatic interaction |
| | G64C_G196C | disulfide |
| | A137C_I267C | disulfide |
| | V175L | cavity filling |
| Combo41-0 | G64C_G196C | disulfide |
| | A137C_I267C | disulfide |
| | V175L | cavity filling |
| Combo42 | L168Q | electrostatic interaction |
| | G64C_G196C | disulfide |
| | A137C_I267C | disulfide |
| | S125P | proline |
| Combo42-0 | G64C_G196C | disulfide |
| | A137C_I267C | disulfide |
| | S125P | proline |
| Combo43 | L168Q | electrostatic interaction |
| | G64C_G196C | disulfide |
| | A137C_I267C | disulfide |
| | S125P | proline |
| | V175L | cavity filling |
| Combo43-0 | G64C_G196C | disulfide |
| | A137C_I267C | disulfide |
| | S125P | proline |
| | V175L | cavity filling |
| Combo44 | L168Q | electrostatic interaction |
| | G64C_G196C | disulfide |
| | A137C_I267C | disulfide |
| | T367R | polar |
| Combo44-0 | G64C_G196C | disulfide |
| | A137C_I267C | disulfide |
| | T367R | polar |
| Combo45 | L168Q | electrostatic interaction |
| | G64C_G196C | disulfide |
| | A137C_I267C | disulfide |
| | K471A | charge ablation |
| | S125P | proline |
| Combo45-0 | G64C_G196C | disulfide |
| | A137C_I267C | disulfide |
| | K471A | charge ablation |
| | S125P | proline |
| Combo46 | L168Q | electrostatic interaction |
| | G64C_G196C | disulfide |
| | A137C_I267C | disulfide |
| | K471A | charge ablation |
| | S125P | proline |
| | T367R | polar |
| | V175L | cavity filling |
| Combo46-0 | G64C_G196C | disulfide |
| | A137C_I267C | disulfide |
| | K471A | charge ablation |
| | S125P | proline |
| | T367R | polar |
| | V175L | cavity filling |
| Combo47 | L168Q | electrostatic interaction |
| | G64C_G196C | disulfide |
| | A137C_I267C | disulfide |
| Combo47-0 | G64C_G196C | disulfide |
| | A137C_I267C | disulfide |
| Combo48 | G64C_G196C | disulfide |
| | A137C_I267C | disulfide |
| | K471A | charge ablation |
| | S125P | proline |
| | L282F | cavity filling |
| | V175L | cavity filling |
| Combo48-1 | L168Q | electrostatic interaction |
| | G64C_G196C | disulfide |
| | A137C_I267C | disulfide |
| | K471A | charge ablation |
| | S125P | proline |
| | L282F | cavity filling |
| | V175L | cavity filling |
| Combo49 | G64C_G196C | disulfide |
| | L147C_A171C | disulfide |
| | K471A | charge ablation |
| | S125P | proline |
| | L282F | cavity filling |
| | V175L | cavity filling |
| Combo49-1 | L168Q | electrostatic interaction |
| | G64C_G196C | disulfide |
| | L147C_A171C | disulfide |
| | K471A | charge ablation |
| | S125P | proline |
| | L282F | cavity filling |
| | V175L | cavity filling |
| Combo50 | G64C_G196C | disulfide |
| | L147C_A171C | disulfide |
| | A137C_I267C | disulfide |
| | K471A | charge ablation |
| | S125P | proline |
| | L282F | cavity filling |
| | V175L | cavity filling |
| Combo50-1 | L168Q | electrostatic interaction |
| | G64C_G196C | disulfide |
| | L147C_A171C | disulfide |
| | A137C_I267C | disulfide |
| | K471A | charge ablation |
| | S125P | proline |
| | L282F | cavity filling |
| | V175L | cavity filling |
| Combo51 | G64C_G196C | disulfide |
| | L147C_A171C | disulfide |
| Combo51-1 | L168Q | electrostatic interaction |
| | G64C_G196C | disulfide |
| | L147C_A171C | disulfide |
| Combo52 | G64C_G196C | disulfide |
| | L147C_A171C | disulfide |
| | V28M | cavity filling |
| | V175L | cavity filling |
| | I201W | cavity filling |
| | L228W | cavity filling |
| | S125P | proline |
| | T367R | polar |
| | K471A | charge ablation |
| Combo52 | L168Q | electrostatic interaction |
| | G64C_G196C | disulfide |
| | L147C_A171C | disulfide |
| | V28M | cavity filling |
| | V175L | cavity filling |
| | I201W | cavity filling |
| | L228W | cavity filling |
| | S125P | proline |
| | T367R | polar |
| | K471A | charge ablation |
| Combo53 | G64C_G196C | disulfide |
| | A137C_I267C | disulfide |
| | V28M | cavity filling |
| | V175L | cavity filling |
| | I201W | cavity filling |
| | L228W | cavity filling |
| | S125P | proline |
| | T367R | polar |
| | K471A | charge ablation |
| Combo53-1 | L168Q | electrostatic interaction |
| | G64C_G196C | disulfide |
| | A137C_I267C | disulfide |
| | V28M | cavity filling |
| | V175L | cavity filling |
| | I201W | cavity filling |
| | L228W | cavity filling |
| | S125P | proline |
| | T367R | polar |
| | K471A | charge ablation |
| Combo54 | G64C_G196C | disulfide |
| | L147C_A171C | disulfide |
| | A137C_I267C | disulfide |
| | V28M | cavity filling |
| | V175L | cavity filling |
| | I201W | cavity filling |
| | L228W | cavity filling |
| | S125P | proline |
| | T367R | polar |
| | K471A | charge ablation |
| Combo54-1 | L168Q | electrostatic interaction |
| | G64C_G196C | disulfide |
| | L147C_A171C | disulfide |
| | A137C_I267C | disulfide |
| | V28M | cavity filling |
| | V175L | cavity filling |
| | I201W | cavity filling |
| | L228W | cavity filling |
| | S125P | proline |
| | T367R | polar |
| | K471A | charge ablation |
| Combo55 | L168Q | electrostatic interaction |
| | G64C_G196C | disulfide |
| | I151C_A171C | disulfide |
| | A137C_I267C | disulfide |
| Combo55-0 | G64C_G196C | disulfide |
| | I151C_A171C | disulfide |
| | A137C_I267C | disulfide |
| Combo56 | L168Q | electrostatic interaction |
| | G64C_G196C | disulfide |
| | I151C_A171C | disulfide |
| | A137C_I267C | disulfide |
| | I231C_G230C | disulfide |
| Combo56-0 | G64C_G196C | disulfide |
| | I151C_A171C | disulfide |
| | A137C_I267C | disulfide |
| | I231C_G230C | disulfide |
| Combo57 | L168Q | electrostatic interaction |
| | G64C_G196C | disulfide |
| | A137C_I267C | disulfide |
| | V28M | cavity filling |
| | V175L | cavity filling |
| | I201W | cavity filling |
| | L228W | cavity filling |
| | R281Y | cavity filling |
| Combo57-0 | G64C_G196C | disulfide |
| | A137C_I267C | disulfide |
| | V28M | cavity filling |
| | V175L | cavity filling |
| | I201W | cavity filling |
| | L228W | cavity filling |
| | R281Y | cavity filling |
| Combo58 | L168Q | electrostatic interaction |
| | G64C_G196C | disulfide |
| | A137C_I267C | disulfide |
| | V28M | cavity filling |
| | V175L | cavity filling |
| | I201W | cavity filling |
| | L282F | cavity filling |
| | L228W | cavity filling |
| | R281Y | cavity filling |
| Combo58-0 | G64C_G196C | disulfide |
| | A137C_I267C | disulfide |
| | V28M | cavity filling |
| | V175L | cavity filling |
| | I201W | cavity filling |
| | L282F | cavity filling |
| | L228W | cavity filling |
| | R281Y | cavity filling |
| Combo 59 | L168Q | electrostatic interaction |
| | I151C_A171C | disulfide |
| | V449C_S457C | interprotomer DS |
| | V28M | cavity filling |
| | V175L | cavity filling |
| | R281Y | cavity filling |
| Combo 59-0 | I151C_A171C | disulfide |
| | V449C_S457C | interprotomer DS |
| | V28M | cavity filling |
| | V175L | cavity filling |
| | R281Y | cavity filling |
| Combo 60 | I151C_A171C | disulfide |
| | S186C_A195C | disulfide |
| | V28M | cavity filling |
| | V175L | cavity filling |
| | R281Y | cavity filling |
| Combo 60-1 | L168Q | electrostatic interaction |
| | I151C_A171C | disulfide |
| | S186C_A195C | disulfide |
| | V28M | cavity filling |
| | V175L | cavity filling |
| | R281Y | cavity filling |
| Combo 61 | L168Q | electrostatic interaction |
| | I151C_A171C | disulfide |
| | L228W | cavity filling |
| | L282F | cavity filling |
| | R821Y | cavity filling |
| | V175L | cavity filling |
| Combo 61-0 | I151C_A171C | disulfide |
| | L228W | cavity filling |
| | L282F | cavity filling |
| | R821Y | cavity filling |
| | V175L | cavity filling |
| Combo 61-0-1 | I151C_A171C | disulfide |
| | L228W | cavity filling |
| | L282F | cavity filling |
| | R821Y | cavity filling |
| | V175L | cavity filling |
| | G200E | cavity filling |
| | I57F | cavity filling |
| Combo 61-0-1-1 | L168Q | electrostatic interaction |
| | I151C_A171C | disulfide |
| | L228W | cavity filling |
| | L282F | cavity filling |
| | R821Y | cavity filling |
| | V175L | cavity filling |
| | G200E | cavity filling |
| | I57F | cavity filling |
| Combo 61-0-2 | I151C_A171C | disulfide |
| | L228W | cavity filling |
| | L282F | cavity filling |
| | R821Y | cavity filling |
| | V175L | cavity filling |
| | G200E | cavity filling |
| | I57F | cavity filling |
| | V449C_S457C | interprotomer DS |
| Combo 61-0-2-1 | L168Q | electrostatic interaction |
| | I151C_A171C | disulfide |
| | L228W | cavity filling |
| | L282F | cavity filling |
| | R821Y | cavity filling |
| | V175L | cavity filling |
| | G200E | cavity filling |
| | I57F | cavity filling |
| | V449C_S457C | interprotomer DS |
| Combo 62 | L168Q | electrostatic interaction |
| | I151C_A171C | disulfide |
| | G200E | cavity filling |
| | I57F | cavity filling |
| Combo 62-0 | I151C_A171C | disulfide |
| | G200E | cavity filling |
| | I57F | cavity filling |
| Combo 62-0-1 | I151C_A171C | disulfide |
| | G200E | cavity filling |
| | I57F | cavity filling |
| | V449C_S457C | interprotomer DS |
| Combo 62-0-1-1 | L168Q | electrostatic interaction |
| | I151C_A171C | disulfide |
| | G200E | cavity filling |
| | I57F | cavity filling |
| | V449C_S457C | interprotomer DS |
| Combo59x | I151C_A171C | disulfide |
| | V449C_S457C | interprotomer DS |
| Combo59x-1 | L168Q | electrostatic interaction |
| | I151C_A171C | disulfide |
| | V449C_S457C | interprotomer DS |

### III. PROTEIN PREPARATION

The protein described herein may be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and HIGH FIVE cells (a clonal isolate derived from the parental Trichoplusia ni BTI-TN-5B1-4 cell line). Examples of suitable mammalian cells include Chinese hamster ovary (CHO) cells, human embryonic kidney cells (HEK293 or Expi 293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx.®. cells), chicken embryonic fibroblasts, chicken embryonic germ cells, quail fibroblasts (e.g., ELL-O), and duck cells. Suitable insect cell expression systems, such as baculovirus-vectored systems, are known to those of skill in the art. Materials and methods for baculovirus/insect cell expression systems are commercially available in kit form. Avian cell expression systems are also known to those of skill in the art. Similarly, bacterial and mammalian cell expression systems are also known in the art.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as PFASTBAC, is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The proteins can be purified using any suitable methods. For example, methods for purifying a protein by immunoaffinity chromatography are known in the art. Suitable methods for purifying desired proteins including precipitation and various types of chromatography, such as hydrophobic interaction, ion exchange, affinity, chelating and size exclusion are known in the art. Suitable purification schemes can be created using two or more of these or other suitable methods. If desired, the protein may include a “tag” that facilitates purification, such as an epitope tag or a histidine tag. Such tagged proteins can be purified, for example from conditioned media, by chelating chromatography or affinity chromatography.

### IV. NUCLEIC ACIDS ENCODING PROTEINS

Also provided are nucleic acid molecules that encode a protein described herein. These nucleic acid molecules include DNA, cDNA, and RNA sequences. Nucleic acid molecules that encode only the ectodomain of the protein are also contemplated. The nucleic acid molecule can be incorporated into a vector, such as an expression vector.

The nucleic acid may be a self-replicating RNA molecule. The nucleic acid may include a modified RNA molecule. Also provided are compositions comprising a nucleic acid described herein.

### V. FORMULATIONS AND METHODS OF USE THEREOF

Provided herein are methods of inducing an immune response against a PIV in a mammal, the methods including administering to the mammal an immunologic composition in an amount effective to induce an immune response, wherein the composition includes an engineered PIV F prefusion protein or a polynucleotide encoding an engineered PIV F prefusion protein. The immune response induced may be a protective immune response, i.e., the response reduces the risk or severity of or clinical consequences of a PIV infection. The immune response can comprise a humoral immune response, a cell-mediated immune response, or both. The immune response may include a T cell response or a B cell response. A cell-mediated immune response can comprise a Helper T-cell (Th) response, a CD8+ cytotoxic T-cell (CTL) response, or both. The humoral immune response may comprise antibody-presenting B cells, and the antibodies may be neutralizing antibodies against PIV. Neutralizing antibodies block viral infection of cells. The immune response may reduce or prevent infection of cells. Neutralizing antibody responses can be complement-dependent or complement-independent. The neutralizing antibody response may be complement-independent. The neutralizing antibody response may be cross-neutralizing; i.e., an antibody generated against an administered composition neutralizes a related PIV virus of a strain other than the strain used in the composition.

The method may involve a single administration of the composition. The method may further include administering to the subject a booster dose of the composition.

A desired response is to inhibit or reduce or prevent PIV infection. A desired response is to reduce PIV viral shedding. The methods may reduce PIV viral shedding in saliva. The reduction in PIV viral shedding in a mammal is as compared to the viral shedding in mammals that were not administered the engineered PIVF protein. The term “viral shedding” is used herein according to its plain ordinary meaning in medicine and virology and refers to the production and release of virus from an infected cell. The virus may be released from a cell of a mammal. Virus may be released into the environment from an infected mammal. Virus may be released from a cell within a mammal. A desired response is to reduce PIV viral titers. The methods may reduce PIV nucleic acids in serum.

The PIV infection, viral shedding, or viral titers do not need to be completely eliminated or reduced or prevented for the method to be effective. For example, administration of an effective amount of the agent can decrease the PIV infection (for example, as measured by infection of cells, or by number or percentage of subjects infected by PIV), viral shedding, or viral titers by a desired amount, for example by at least 50%, at least 60%, at least 70%, at least 80%, at least 90%, at least 95%, at least 98%, or even at least 100% (elimination or prevention of detectable PIV infection, viral shedding, or viral titers), as compared to a suitable control.

The engineered proteins described herein can be delivered directly as a component of an immunogenic composition or a vaccine. Alternatively, nucleic acids that encode the proteins described herein can be administered to produce the protein or immunogenic fragment in vivo. Protein formulations, recombinant nucleic acids (e.g., DNA, RNA, mRNA, self-replicating RNA, or any variation thereof) and/or viral vectors (e.g., live, single-round, non-replicative assembled virions, or otherwise virus-like particles, or alphavirus VRP) that contain sequences encoding the engineered proteins provided herein may be included in an immunogenic composition or a vaccine. Such compositions may produce the proteins described herein upon translation of an open reading frame, which may be codon-optimized. A composition may include at least one RNA polynucleotide encoding at least one PIV F antigenic polypeptide or an immunogenic fragment thereof and at least one 5′ terminal cap. A 5′ terminal cap may be 7mG(5′)ppp(5′)NlmpNp.

In the case that a nucleic acid molecule encoding an engineered PIV F protein is used in a pharmaceutical composition, the nucleic acid molecule may comprise or consist of deoxyribonucleotides and/or ribonucleotides, or analogs thereof, covalently linked together. A nucleic acid molecule as described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. A nucleic acid molecule may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A nucleic acid molecule is composed of a specific sequence of four nucleotide bases: adenine (A), cytosine (C), guanine (G), thymine (T), and uracil (U) for thymine when the polynucleotide is RNA. Thus, the term “nucleic acid sequence” is the alphabetical representation of a nucleic acid molecule. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The at least one polynucleotide may have at least one chemical modification. The at least one polynucleotide may further include a second chemical modification. The polynucleotide may be RNA. The at least one polynucleotide having at least one chemical modification may have a 5′ terminal cap. The at least one chemical modification may be selected from pseudouridine, N1-methylpseudouridine, N1-ethylpseudouridine, N1-ethylpseudouridine, 2-thiouridine, 4′-thiouridine, 5-methylcytosine, 2-thio-1-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-0-methyl uridine. At least 80% (e.g., 85%, 90%, 95%, 98%, 99%, 100%) of the uracils in the open reading frame may have a chemical modification, optionally wherein the composition is formulated in a lipid nanoparticle. All of the uracils in the open reading frame may have a chemical modification. The chemical modification may be in the 5-position of the uracil. The chemical modification may be an N1-methyl pseudouridine.

The nucleic acids of the present disclosure may comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH3 (“OMe” or “O-methyl”), and 2′-O(CH2)2OCH3 (“MOE”). In certain embodiments, sugar substituents at the 2′ position is selected from allyl, amino, azido, thio, O-allyl, O—C1-C10 alkyl, O—C1-C10 substituted alkyl; OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF3, OCF3, O, S, or N(Rm)-alkyl; O, S, or N(Rm)-alkenyl; 0, S or N(Rm)-alkynyl; O-alkylenyl-O-alkyl, alkynyl, alkaryl, aralkyl, O-alkaryl, O-aralkyl, O(CH2)2SCH3, O(CH2)2-O—N(Rm)(Rn) or O—CH2-C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (NO2), thiol, thioalkoxy (S-alkyl), halogen, alkyl, aryl, alkenyl and alkynyl.

In some embodiments, a 2′-substituted nucleoside comprises a 2′-substituent group selected from F, NH2, N3, OCF3, O—CH3, O(CH2)3NH2, CH2-CH═CH2, O—CH2-CH═CH2, OCH2CH2OCH3, O(CH2)2SCH3, O—(CH2)2-O—N(Rm)(Rn), O(CH2)2O(CH2)2N(CH3)2, and N-substituted acetamide (0-CH2-C(═O)—N(Rm)(Rn) where each Rm and Rn is, independently, H, an amino protecting group or substituted or unsubstituted C1-C10 alkyl.

In some embodiments, a 2′-substituted nucleoside comprises a sugar moiety comprising a 2′-substituent group selected from F, OCF3, O—CH3, OCH2CH2OCH3, O(CH2)2SCH3, O(CH2)2-O—N(CH3)2, —O(CH2)2O(CH2)2N(CH3)2, and O—CH2-C(═O)—N(H)CH3.

In some embodiments, a 2′-substituted nucleoside comprises a sugar moiety comprising a 2′-substituent group selected from F, O—CH3, and OCH2CH2OCH3.

In some embodiments, nucleosides of the present disclosure comprise one or more unmodified nucleobases. In certain embodiments, nucleosides of the present disclosure comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States Patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution, or cellular uptake of the oligonucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996). In some aspects, a nucleic acid molecule encoding an engineered PIV F protein is a modified RNA, such as, for example, a modified mRNA. Modified (m)RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In some embodiments, the (m)RNA molecules used herein may have the uracils replaced with psuedouracils such as 1-methyl-3′-pseudouridylyl bases. In some embodiments, some of the uracils are replaced, but in other embodiments, all of the uracils have been replaced. The (m)RNA may comprise a 5′ cap, a 5′ UTR element, an optionally codon optimized open reading frame, a 3′ UTR element, and a poly(A) sequence and/or a polyadenylation signal.

The nucleic acid molecule, whether native or modified, may be delivered as a naked nucleic acid molecule or in a delivery vehicle, such as a lipid nanoparticle. A lipid nanoparticle may comprise one or more nucleic acids present in a weight ratio to the lipid nanoparticles from about 5:1 to about 1:100. In some embodiments, the weight ratio of nucleic acid to lipid nanoparticles is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any value derivable therein.

In some embodiments, the lipid nanoparticles used herein may contain one, two, three, four, five, six, seven, eight, nine, or ten lipids. These lipids may include triglycerides, phospholipids, steroids or sterols, PEGylated lipids, or a group with an ionizable group such as an alkyl amine and one or more hydrophobic groups such as C6 or greater alkyl groups.

In some aspects of the present disclosure, the lipid nanoparticles are mixed with one or more steroid or a steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms.

In some aspects of the present disclosure, the lipid nanoparticles are mixed with one or more PEGylated lipids (or PEG lipids). In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In some aspects of the present disclosure, the lipid nanoparticles are mixed with one or more phospholipids. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some aspects of the present disclosure, lipid nanoparticle containing compounds containing lipophilic and cationic components, wherein the cationic component is ionizable, are provided. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C6-C24 alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some aspects of the present disclosure, composition containing compounds containing lipophilic and cationic components, wherein the cationic component is ionizable, are provided. In some embodiments, ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C6-C24 alkyl or alkenyl groups, but may have at least 1 or more that 6 tails.

In some embodiments, the amount of the lipid nanoparticle with the nucleic acid molecule encapsulated in the pharmaceutical composition is from about 0.1% w/w to about 50% w/w, from about 0.25% w/w to about 25% w/w, from about 0.5% w/w to about 20% w/w, from about 1% w/w to about 15% w/w, from about 2% w/w to about 10% w/w, from about 2% w/w to about 5% w/w, or from about 6% w/w to about 10% w/w. In some embodiments, the amount of the lipid nanoparticle with the nucleic acid molecule encapsulated in the pharmaceutical composition is from about 0.1% w/w, 0.25% w/w, 0.5% w/w, 1% w/w, 2.5% w/w, 5% w/w, 7.5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, to about 95% w/w, or any range derivable therein.

In some aspects, the present disclosure comprises one or more sugars formulated into pharmaceutical compositions. In some embodiments, the sugars used herein are saccharides. These saccharides may be used to act as a lyoprotectant that protects the pharmaceutical composition from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol.

In some embodiments, the amount of the sugar in the pharmaceutical composition is from about 25% w/w to about 98% w/w, 40% w/w to about 95% w/w, 50% w/w to about 90% w/w, 50% w/w to about 70% w/w, or from about 80% w/w to about 90% w/w. In some embodiments, the amount of the sugar in the pharmaceutical composition is from about 10% w/w, 15% w/w, 20% w/w, 25% w/w, 30% w/w, 35% w/w, 40% w/w, 45% w/w, 50% w/w, 52.5% w/w, 55% w/w, 57.5% w/w, 60% w/w, 62.5% w/w, 65% w/w, 67.5% w/w, 70% w/w, 75% w/w, 80% w/w, 82.5% w/w, 85% w/w, 87.5% w/w, 90% w/w, to about 95% w/w, or any range derivable therein.

In some embodiments, the pharmaceutically acceptable polymer is a copolymer. The pharmaceutically acceptable polymer may further comprise one, two, three, four, five, or six subunits of discrete different types of polymer subunits. These polymer subunits may include polyoxypropylene, polyoxyethylene, or a similar subunit. In particular, the pharmaceutically acceptable polymer may comprise at least one hydrophobic subunit and at least one hydrophilic subunit. In particular, the copolymer may have hydrophilic subunits on each side of a hydrophobic unit. The copolymer may have a hydrophilic subunit that is polyoxyethylene and a hydrophobic subunit that is polyoxypropylene.

In some embodiments, expression cassettes are employed to express a PIV F protein, either for subsequent purification and delivery to a cell/subject, or for use directly in a viral-based delivery approach. Provided herein are expression vectors which contain one or more nucleic acids encoding a PIV F protein.

Expression requires that appropriate signals be provided in the vectors and include various regulatory elements such as enhancers/promoters from both viral and mammalian sources that drive expression of the engineered PIV F protein in cells. Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase IL. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 hp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promoters such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ P, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), ec-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), SV40, polyoma, retroviruses, papilloma virus, hepatitis B virus, human immunodeficiency virus, cytomegalovirus (CMV), and gibbon ape leukemia virus.

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One method for in vivo delivery involves the use of an adenovirus expression vector. “Adenovirus expression vector” is meant to include those constructs containing adenovirus sequences sufficient to (a) support packaging of the construct and (b) to express an engineered PIV F protein that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (EIA and ElB) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

The adenoviruses of the disclosure are replication defective, or at least conditionally replication defective. The adenovirus may be of any of the 42 different known serotypes or subgroups A-F. Adenovirus type 5 of subgroup C is one exemplary starting material that may be used to obtain the conditional replication-defective adenovirus vector for use in the present disclosure.

Other viral vectors may be employed as expression constructs in the present disclosure. Vectors derived from viruses such as vaccinia virus, adeno-associated virus (AAV) and herpesviruses may be employed. They offer several attractive features for various mammalian cells.

In embodiments, particular embodiments, the vector is an AAV vector. AAV is a small virus that infects humans and some other primate species. AAV is not currently known to cause disease. The virus causes a very mild immune response, lending further support to its apparent lack of pathogenicity. In many cases, AAV vectors integrate into the host cell genome, which can be important for certain applications, but can also have unwanted consequences. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. This feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) proteins can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

Each of the immunogenic compositions discussed herein may be used alone or in combination with one or more other antigens, the latter either from the same viral pathogen or from another pathogenic source or sources. These compositions may be used for prophylactic (to prevent infection) or therapeutic (to treat disease after infection) purposes.

The term “pharmaceutically acceptable” may mean approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and can preferably include an adjuvant. Water is a particular carrier when the pharmaceutical composition is administered by injections, such an intramuscular injection. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The immunogenic composition may include a diluent, such as water, saline, glycerol, ethanol, etc. Additionally, auxiliary substances, such as wetting or emulsifying agents, pH buffering substances, and the like, may be present in such vehicles. The immunogenic composition may contain one or more salts. The salts may be an inorganic potassium or sodium salt such as potassium chloride, sodium chloride, potassium phosphate dibasic, potassium phosphate monobasic, sodium phosphate dibasic, or sodium phosphate monobasic. The immunogenic composition may comprise one or more phosphate salts such to generate a phosphate buffer solution. The phosphate buffer solution may comprise each of the phosphates to buffer a solution to a pH from about 6.8, 6.9, 7.0, 7.1, 7.2, 7.3, 7.4, 7.5, 7.6, 7.7, 7.8, 7.9, or 8.0, or any range derivable therein.

The immunogenic composition may include an adjuvant. Exemplary adjuvants to enhance effectiveness of the composition include: (1) aluminum salts (alum), such as aluminum hydroxide, aluminum phosphate, aluminum sulfate, etc.; (2) oil-in-water emulsion formulations (with or without other specific adjuvants such as muramyl peptides (see below) or bacterial cell wall components), such as for example (a) MF59 (PCT Publ. No. WO 90/14837), containing 5% Squalene, 0.5% TWEEN 80, and 0.5% Span 85 formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DETOX™); (3) saponin adjuvants, such as QS-21, STIMULON™ (Cambridge Bioscience, Worcester, Mass.), which may be used or particles generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), etc.; (6) toll-like receptor agonists; and (7) other substances that act as adjuvants to enhance the effectiveness of the composition. The composition may not include an adjuvant. The composition may further include a lipid nanoparticle. The composition may be formulated in a nanoparticle. The composition may further include a cationic or polycationic compound, including protamine or other cationic peptides or proteins, such as poly-L-lysine (PLL).

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions described herein may include an immunologically effective amount of the polypeptide or polynucleotide, as well as any other of the above-mentioned components, as needed. By “immunologically effective amount,” it is meant that the administration of that amount to an individual, either in a single dose or as part of a series, is effective for eliciting an immune response. The immune response elicited may be sufficient, for example, for treatment and/or prevention and/or reduction in incidence of illness, infection or disease. This amount varies depending upon the health and physical condition of the individual to be treated, the taxonomic group of individual to be treated (e.g., nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

Any suitable route of administration can be used. For example, engineered proteins or nucleic acids encoding engineered proteins of the present disclosure, as described herein, can be formulated for parenteral administration, e.g., formulated for injection via the intradermal, intravenous, intraarterial, intramuscular, subcutaneous, intra-tumoral or even intraperitoneal routes. Particularly preferred routes of administration include intramuscular, intradermal and subcutaneous injection. The formulation could alternatively be administered by a topical route directly to the mucosa, for example by nasal drops, inhalation, or by nebulizer.

Compositions can be administered according to any suitable schedule. Dosage treatment may be a single dose schedule or a multiple dose schedule. Multiple doses may be used in a primary immunization schedule and/or in a booster immunization schedule. In a multiple dose schedule the various doses may be given by the same or different routes. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.). The immunogenic composition may be administered in conjunction with other immunoregulatory agents.

The compositions disclosed herein may be used to treat both children and adults. Thus, a human subject may be less than 1 year old, 1-5 years old, 5-16 years old, 16-55 years old, 55-65 years old, or at least 65 years old.

### VI. ANTIBODIES AND DIAGNOSTIC USES

The polypeptides described above may be used to produce antibodies, both polyclonal and monoclonal. If polyclonal antibodies are desired, a selected mammal (e.g., mouse, rabbit, goat, guinea pig, horse, etc.) is immunized with an immunogenic polypeptide bearing a PIV F prefusion epitope(s). Serum from the immunized animal is collected and treated according to known procedures. If serum containing polyclonal antibodies to a PIV F prefusion epitope contains antibodies to other antigens, the polyclonal antibodies can be purified by immunoaffinity chromatography. Techniques for producing and processing polyclonal antisera are known in the art.

Monoclonal antibodies directed against PIV F prefusion epitopes can also be readily produced by one skilled in the art. The general methodology for making monoclonal antibodies by hybridomas is known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against PIV F prefusion epitopes can be screened for various properties; i.e., for isotype, epitope affinity, etc.

Antibodies, both monoclonal and polyclonal, which are directed against PIV F prefusion epitopes are particularly useful in diagnosis, and those which are neutralizing are useful in passive immunotherapy. Monoclonal antibodies, in particular, may be used to raise anti-idiotype antibodies.

Both the polypeptides which react immunologically with serum containing PIV F antibodies, and the antibodies raised against these polypeptides, may be useful in immunoassays to detect the presence of PIV F antibodies, or the presence of the virus, in biological samples, including for example, blood or serum samples. Design of the immunoassays is subject to a great deal of variation, and a variety of these are known in the art. For example, the immunoassay may utilize the polypeptide having the sequence set forth in any one of SEQ ID NOs: 1-7.

Alternatively, the immunoassay may use a combination of viral antigens derived from the polypeptides described herein. It may use, for example, a monoclonal antibody directed towards at least one polypeptide described herein, a combination of monoclonal antibodies directed towards the polypeptides described herein, monoclonal antibodies directed towards different viral antigens, polyclonal antibodies directed towards the polypeptides described herein, or polyclonal antibodies directed towards different viral antigens. Protocols may be based, for example, upon competition, or direct reaction, or may be sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the engineered PIV F proteins containing PIV F prefusion epitopes or antibodies directed against epitopes in suitable containers, along with the remaining reagents and materials required for the conduct of the assay, as well as a suitable set of assay instructions.

The polynucleotide probes can also be packaged into diagnostic kits. Diagnostic kits include the probe DNA, which may be labeled; alternatively, the probe DNA may be unlabeled and the ingredients for labeling may be included in the kit. The kit may also contain other suitably packaged reagents and materials needed for the particular hybridization protocol, for example, standards, as well as instructions for conducting the test.

### VII. IMMUNODETECTION METHODS

The present disclosure concerns immunodetection methods for binding, purifying, removing, quantifying and otherwise generally detecting PIV F protein. While such methods can be applied in a traditional sense, another use will be in quality control and monitoring of vaccine stocks, where antibodies according to the present disclosure can be used to assess the amount or integrity (i.e., long term stability) of antigens. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of PIV F protein also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing PIV F protein, and contacting the sample with a first antibody in accordance with the present disclosure, as the case may be, under conditions effective to allow the formation of immunocomplexes.

These methods include methods for detecting or purifying PIV F protein from a sample. The antibody will preferably be linked to a solid support, such as in the form of a column matrix, and the sample suspected of containing the PIV F protein will be applied to the immobilized antibody. The unwanted components will be washed from the column, leaving the PIV F protein-expressing cells immunocomplexed to the immobilized antibody, which is then collected by removing the organism or antigen from the column.

The immunobinding methods also include methods for detecting and quantifying the amount of PIV F protein or related components in a sample and the detection and quantification of any immune complexes formed during the binding process. Here, one would obtain a sample suspected of containing PIV F protein and contact the sample with an antibody that binds PIV F protein or components thereof, followed by detecting and quantifying the amount of immune complexes formed under the specific conditions. In terms of antigen detection, the biological sample analyzed may be any sample that is suspected of containing PIV F protein, such as a tissue section or specimen, a homogenized tissue extract, a biological fluid (e.g., a nasal swab), including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to PIV F protein. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the PIV F protein is added to the wells. After binding and washing to remove non-specifically bound immune complexes, the bound antigen may be detected. Detection may be achieved by the addition of another anti-PIV F protein antibody that is linked to a detectable label. This type of ELISA is a simple “sandwich ELISA.” Detection may also be achieved by the addition of a second anti-PIV F protein antibody, followed by the addition of a third antibody that has binding affinity for the second antibody, with the third antibody being linked to a detectable label.

In another exemplary ELISA, the samples suspected of containing the PIV F protein (e.g., potentially infected cells) are immobilized onto the well surface and then contacted with the anti-PIV F protein antibodies of the disclosure. After binding and washing to remove non-specifically bound immune complexes, the bound anti-PIV F protein antibodies are detected. Where the initial anti-PIV F protein antibodies are linked to a detectable label, the immune complexes may be detected directly. Again, the immune complexes may be detected using a second antibody that has binding affinity for the first anti-PIV F protein antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probing. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### D. Immunodetection Kits

In still further embodiments, the present disclosure concerns immunodetection kits for use with the immunodetection methods described above. As the antibodies may be used to detect PIV F protein, the antibodies may be included in the kit. The immunodetection kits will thus comprise, in suitable container means, a first antibody that binds to an PIV F protein, and optionally an immunodetection reagent.

In certain embodiments, the antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of PIV F protein, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### E. Flow Cytometry and FACS

The antibodies of the present disclosure may also be used in flow cytometry or FACS. Flow cytometry is a laser- or impedance-based technology employed in many detection assays, including cell counting, cell sorting, biomarker detection and protein engineering. The technology suspends cells in a stream of fluid and passing them through an electronic detection apparatus, which allows simultaneous multiparametric analysis of the physical and chemical characteristics of up to thousands of particles per second. Flow cytometry is routinely used in the diagnosis disorders, especially blood cancers, but has many other applications in basic research, clinical practice and clinical trials.

Fluorescence-activated cell sorting (FACS) is a specialized type of cytometry. It provides a method for sorting a heterogenous mixture of biological cells into two or more containers, one cell at a time, based on the specific light scattering and fluorescent characteristics of each cell. In general, the technology involves a cell suspension entrained in the center of a narrow, rapidly flowing stream of liquid. The flow is arranged so that there is a large separation between cells relative to their diameter. A vibrating mechanism causes the stream of cells to break into individual droplets. Just before the stream breaks into droplets, the flow passes through a fluorescence measuring station where the fluorescence of each cell is measured. An electrical charging ring is placed just at the point where the stream breaks into droplets. A charge is placed on the ring based immediately prior to fluorescence intensity being measured, and the opposite charge is trapped on the droplet as it breaks form the stream. The charged droplets then fall through an electrostatic deflection system that diverts droplets into containers based upon their charge.

In certain embodiments, to be used in flow cytometry or FACS, the antibodies of the present disclosure are labeled with fluorophores and then allowed to bind to the cells of interest, which are analyzed in a flow cytometer or sorted by a FACS machine.

### VIII. DEFINITIONS

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise. As used herein the specification, “a” or “an” may mean one or more. As used herein in the claim(s), when used in conjunction with the word “comprising,” the words “a” or “an” may mean one or more than one.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2.

IgA is similarly subdivided into subclasses including, but not limited to, IgA 1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

Definitive delineation of a CDR and identification of residues comprising the binding site of an antibody may be accomplished by solving the structure of the antibody and/or solving the structure of the antibody-ligand complex, which can be accomplished by any of a variety of techniques known to those skilled in the art, such as X-ray crystallography. Various methods of analysis may be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgAQ1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itself. Antigens are usually proteins and polysaccharides, less frequently also lipids. As used herein, antigens also include immunogens and haptens.

An “Fc” region comprises two heavy chain fragments comprising the CH1 and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the PIV F protein specific antibodies of the present disclosure are specific to PIV F protein. The antibody that binds to PIV F protein may have a dissociation constant (Kd) of ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻³M, e.g., from 10⁻⁹M to 10⁻¹³ M).

The term “compete” when used in the context of antigen binding proteins (e.g., antibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen (e.g., PIV F protein or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually, the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acids in length. Two antibodies may bind the same epitope within an antigen if they exhibit competitive binding for the antigen.

A useful measure of antibody potency in the art is “50% neutralization titer.” Another useful measure of antibody potency is any one of the following: a “60% neutralization titer”; a “70% neutralization titer”; a “80% neutralization titer”; and a “90% neutralization titer.” To determine, for example, a 50% neutralizing titer, serum from immunized animals is diluted to assess how dilute serum can be yet retain the ability to block entry of 50% of infectious viruses into cells. For example, a titer of 700 means that serum retained the ability to neutralize 50% of infectious virus after being diluted 700-fold. Thus, higher titers indicate more potent neutralizing antibody responses. The titer may be in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50%, 60%, 70%, 80%, or 90% neutralization titer range can have an upper limit of about 400, about 600, about 800, about 1000, about 1500, about 2000, about 2500, about 3000, about 3500, about 4000, about 4500, about 5000, about 5500, about 6000, about 6500, about 7000, about 8000, about 9000, about 10000, about 1 1000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 6500. “About” means plus or minus 10% of the recited value.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) may be also used by the algorithm.

Examples of parameters that can be employed in determining percent identity for polypeptides or nucleotide sequences using the GAP program can be found in Needleman et al., 1970, J. Mol. Biol. 48:443-453.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally occurring amino acid and polymers. The terms “polypeptide” and “protein” specifically encompass PIV F protein binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Fragments may be about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a PIV F protein-binding antibody, useful fragments include but are not limited to a CDR region, a variable domain of a heavy and/or light chain, a portion of an antibody chain or just its variable region including two CDRs, and the like.

The pharmaceutically acceptable carriers are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, goat, swine, sheep, horse or primate). A human includes pre- and post-natal forms. A subject may be a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat viral infection.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The sequence used for the hPIV3 F ectodomains designs contains residues 19-481 of SEQ ID NO: 1. For the screening of single substitution designs, a substitution of L168Q was included to increase solubility and provide for more consistent purification. The GCN4 CC tri2 trimerization domain was included. The construct further included an HRV3C protease recognition site, an octa-histidine tag, and a tandem Twin-Strep-tag, cloned into the mammalian expression plasmid paH. The sequence of this base construct with the L168Q substitution is provided in SEQ ID NO: 8, with position 141 being lysine.

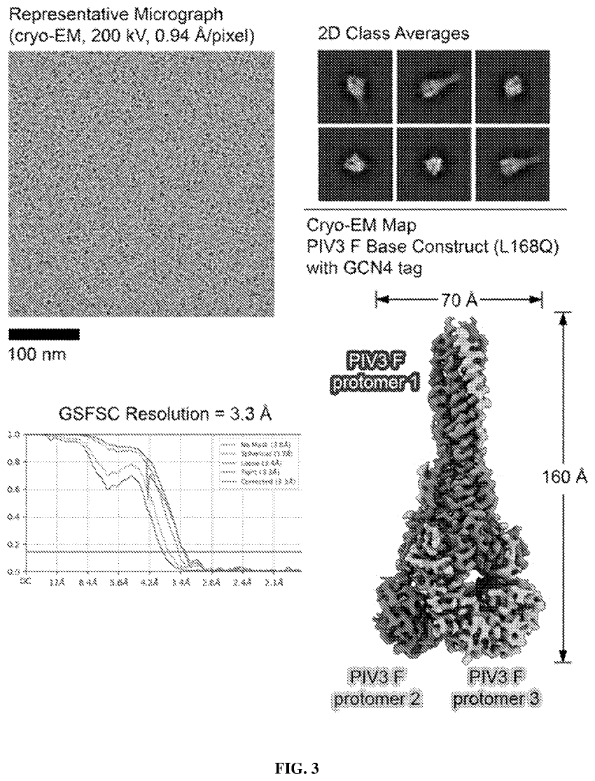

Using structure-based design, substitutions that were aimed at favoring the stability of the prefusion structure were introduced into the base construct (PIV3F L168Q ectodomain with a GCN4 trimerization tag, which adopts the postfusion conformation, FIG. 3; SEQ ID NO: 8 with position 141 being lysine). Pairs of core-facing residues less than 5 Å apart were replaced with aromatic sidechains or pairs of aromatic and positively charged sidechains to favor pi-pi or pi-cation interactions, respectively. Alternatively, residues were replaced with extended or bulkier hydrophobic sidechains in efforts to fill pre-existing internal cavities. Disulfide bonds were designed to increase overall stability or prevent formation of the postfusion conformation. The charged or polar substitutions were aimed to establish hydrogen bonds or salt bridges with the native residues that were predicted to be within 4.0 Å.

Single substitution designs were carried forward into a combinatorial round of screening. For this round, the L168Q substitution from the first round was converted back to leucine. For some combinatorial variants, the L168Q substitution was later re-introduced. In addition, the foldon trimerization motif of T4 fibritin was included between the GCN4 CC tri2 trimerization domain and the HRV3C protease recognition site (SEQ ID NO: 11 with position 141 being lysine).

Exemplary single substitution designs are provided in Table 1. Exemplary combinations of substitutions are provided in Table 2.

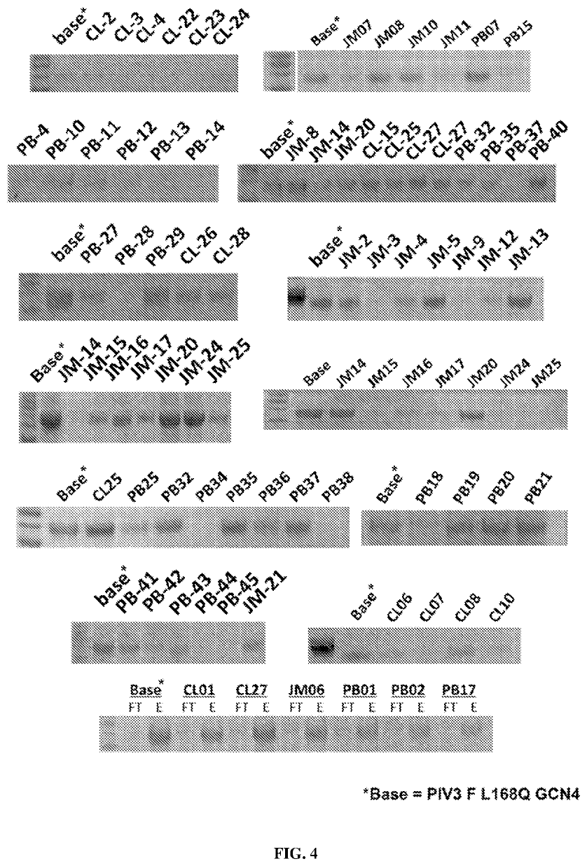

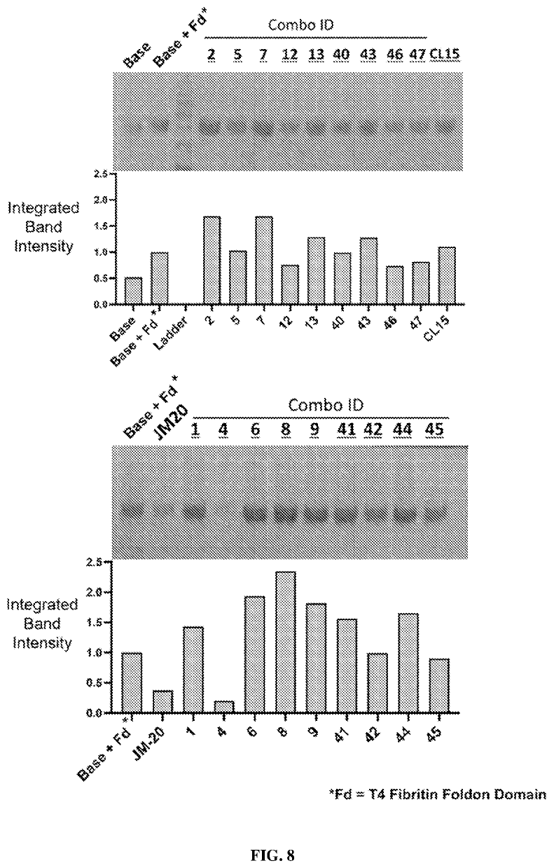

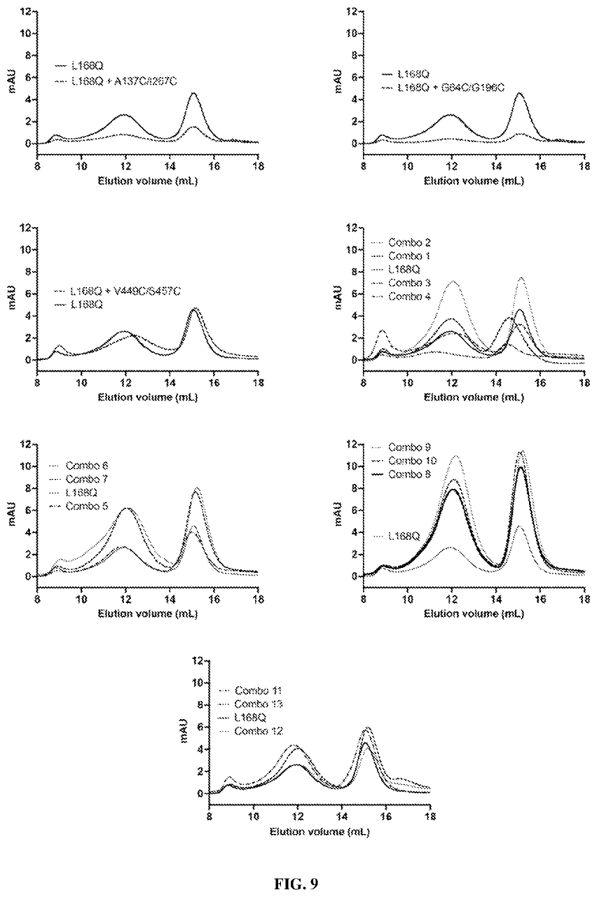

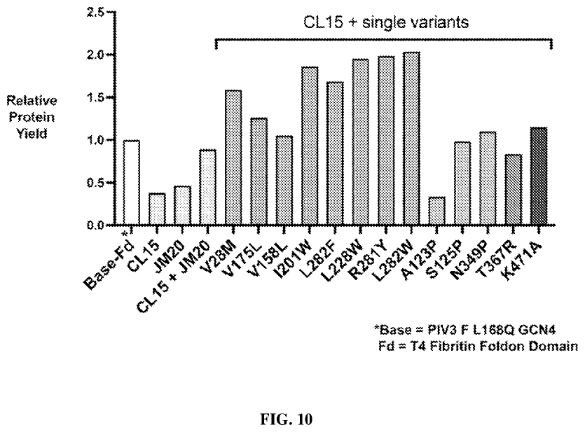

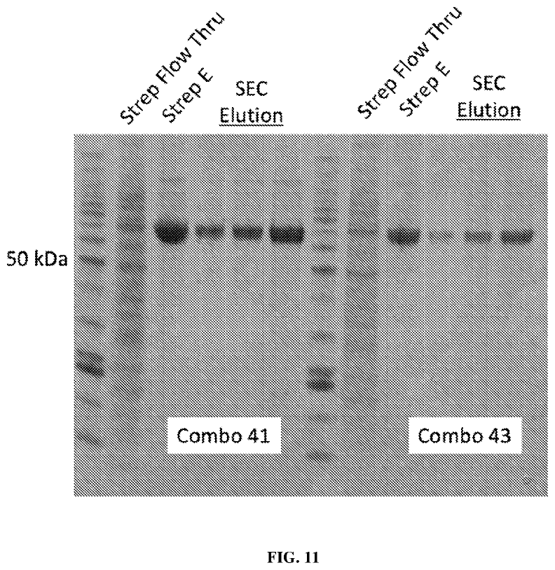

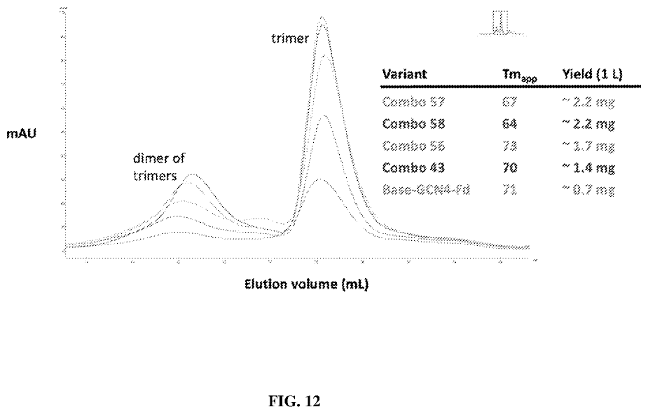

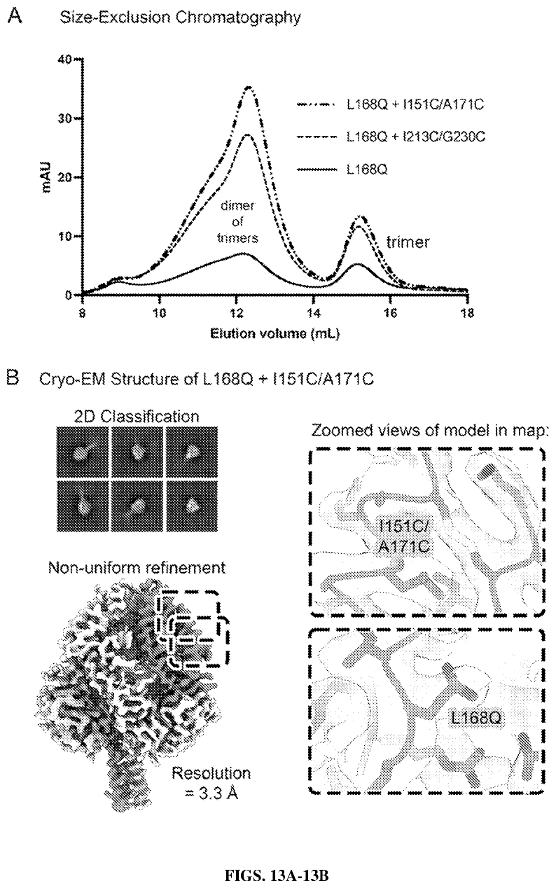

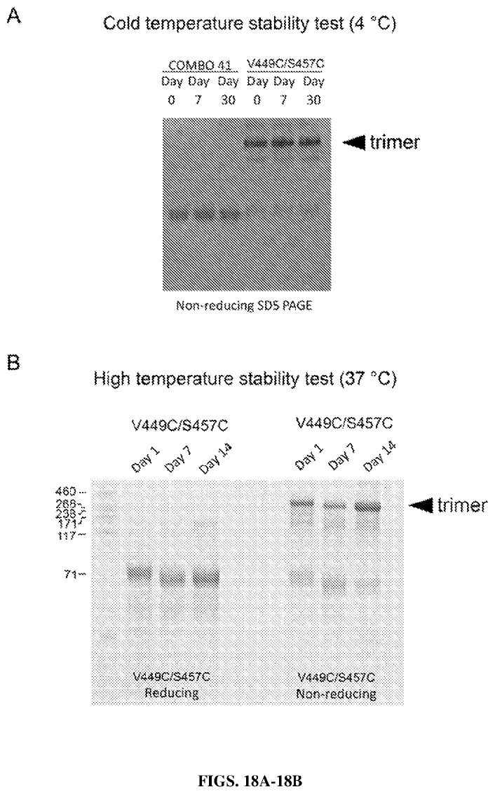

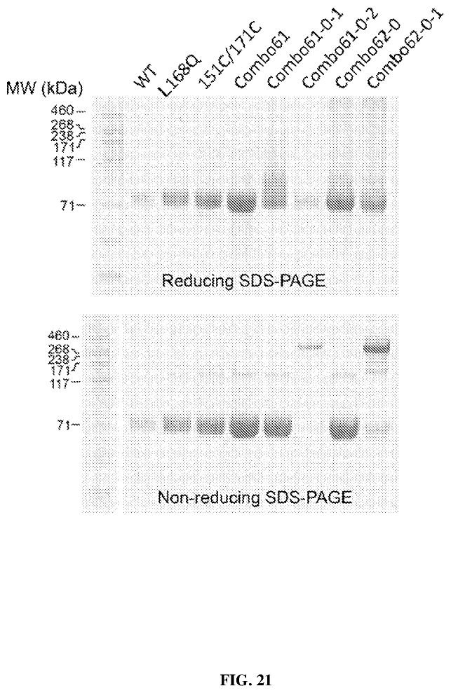

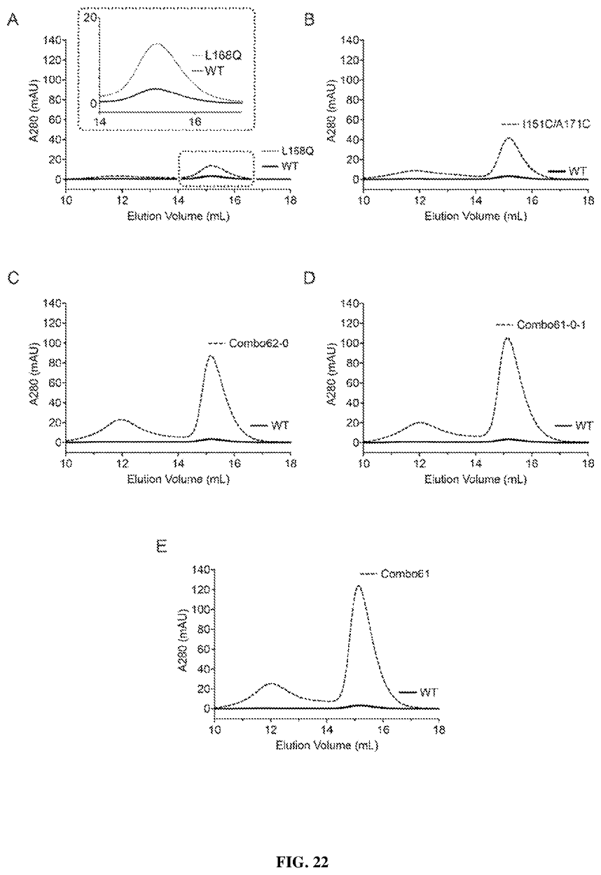

Plasmids encoding hPIV3 F variants were transiently transfected into FreeStyle293F cells (Thermo Fisher) using polyethyleneimine, with 5 μM kifunensine being added 3 h post-transfection. Cultures were grown for 4-6 days, and culture supernatant was separated via centrifugation and passage through a 0.22 m filter. Protein was purified from supernatants using StrepTactin resin (IBA). hPIV3 F variants were further purified by size-exclusion chromatography (SEC) using a Superose 6 10/300 column (GE Healthcare) in a buffer composed of 2 mM Tris pH 8.0, 200 mM NaCl and 0.02% NaN₃. For initial purification and characterization, single-substitution and combinatorial variants were purified from 40 mL cultures. The protein purity, monodispersity and expression level were determined by SDS-PAGE (e.g., FIGS. 4, 8, 10, 11, 17, 18, and 21) and SEC (e.g., FIGS. 9, 12, 13A, and 22). The first peak corresponds to multimers of trimeric post-fusion hPIV3 F; the second peak corresponds to monomeric hPIV3 F trimers.

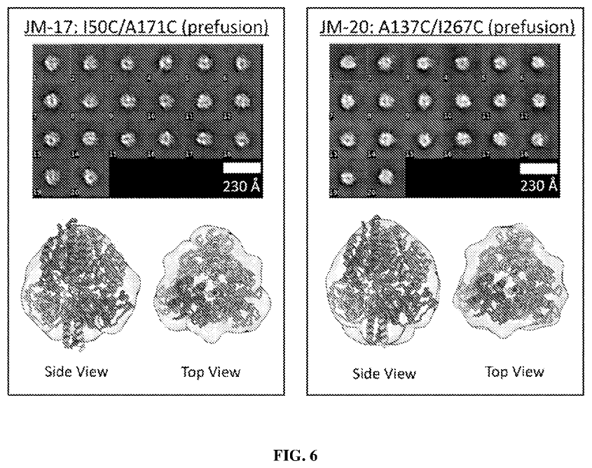

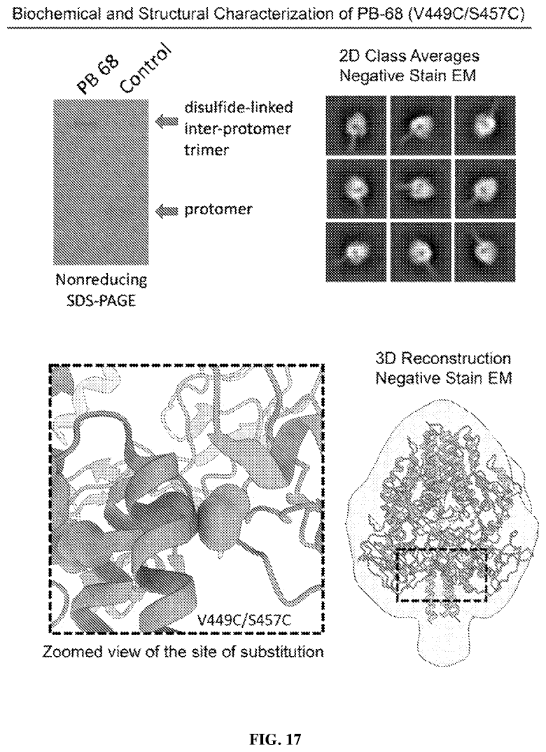

Negative stain electron microscopy (nsEM) analysis was performed on some of the hPIV3 F variants. Purified hPIV3 F variants were diluted to a concentration of 0.06 mg/mL in 2 mM Tris pH 8.0, 200 mM NaCl and 0.02% NaN₃. Each protein was deposited on a CF-400-CU grid (Electron Microscopy Sciences) that had been plasma cleaned for 30 seconds in a Solarus 950 plasma cleaner (Gatan) with a 4:1 ratio of O₂/H₂ and stained using 2% (w/v) uranyl acetate. Grids were imaged at a magnification of 60,000× (corresponding to a calibrated pixel size of 3.6 Å/pix) in a 2010f TEM (Japan Electron Optics Laboratory) operating at 200 kV and equipped with a OneView camera (Gatan). Exemplary data are shown in FIGS. 6 and 17.

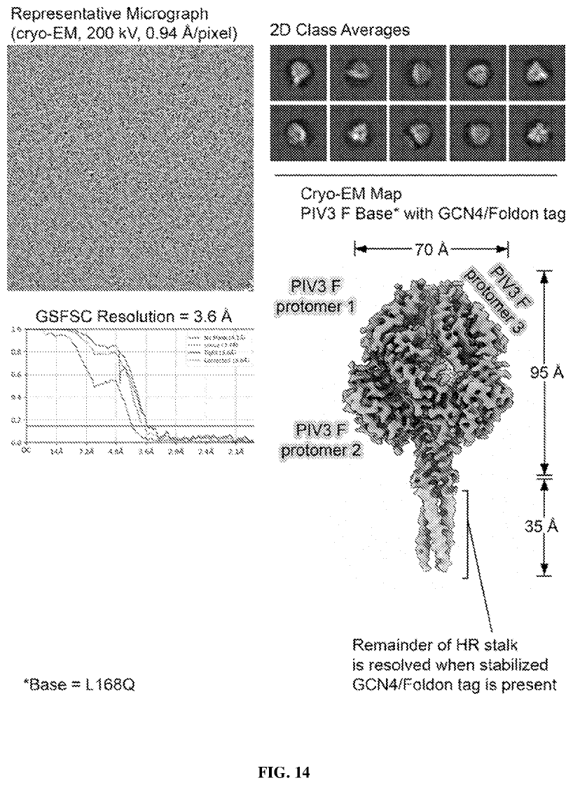

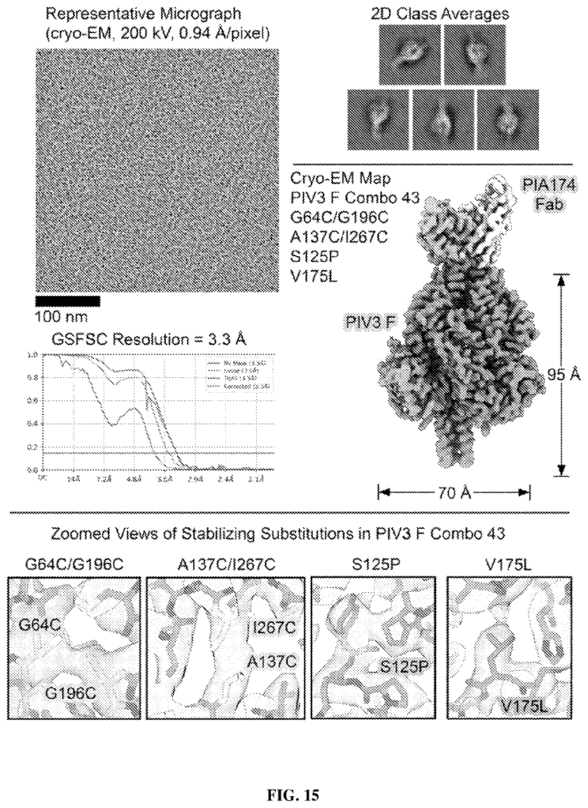

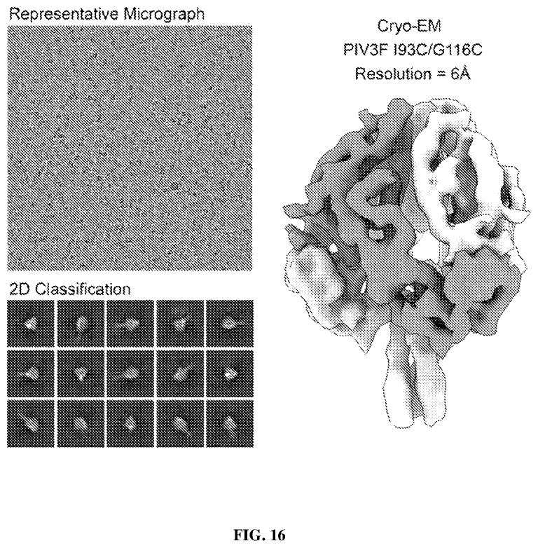

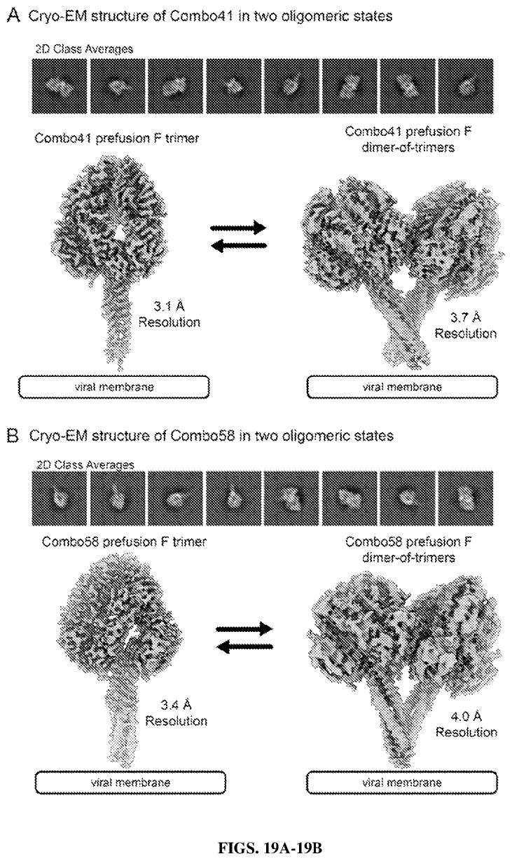

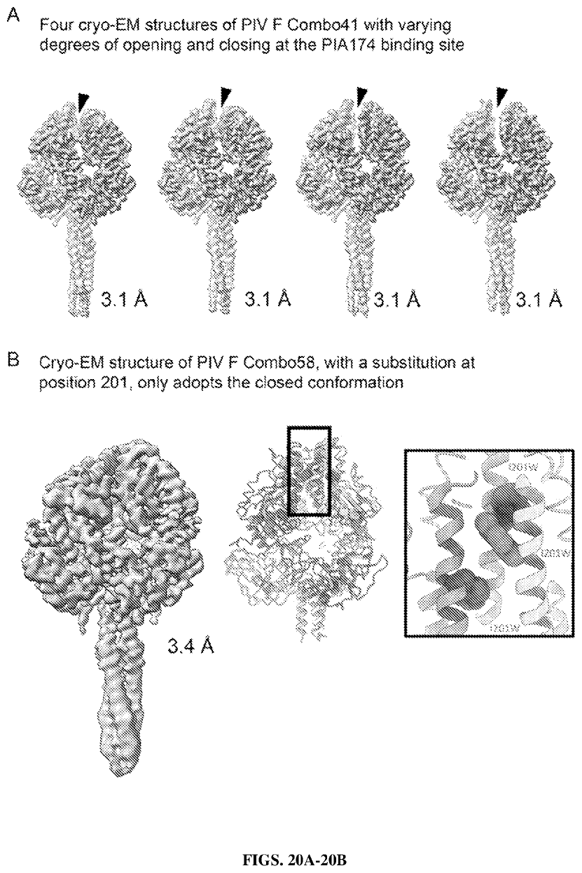

To confirm that stabilizing substitutions did not lead to any unintended conformational changes, the cryo-EM structures of various designs were determined (FIGS. 3, 13B, 14-16, 19, and 20). Purified hPIV3 F variants were diluted to a concentrations ranging from 1-3 mg/mL in 2 mM Tris pH 8.0, 200 mM NaCl, 0.02% NaN₃ and applied to plasma-cleaned CF-400 1.2/1.3 grids or UltrAuFoil 1.2/1.3 grids before being blotted for 3-6 seconds in a Vitrobot Mark IV (ThermoFisher) and plunge frozen into liquid ethane. Micrographs were collected from a single grid using either (i) a Titan Krios (ThermoFisher) equipped with a K3 direct electron detector (Gatan) or (ii) a Glacios (Thermofisher) equipped with a Falcon IV. Data were collected at a calibrated magnification of 0.83 Å/pix for the Krios and 0.94 Å/pix for the Falcon 4.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
International Patent Publication WO 2018/081289
International Patent Publication WO 2022/207839
U.S. Patent Publication 2022/0024987

1. An engineered protein comprising a parainfluenza virus fusion protein (PIV F) ectodomain having at least 90% sequence identity to amino acids 19-481 of SEQ ID NO: 1 or 2, said engineered protein comprising at least one substitution or set of substitutions selected from the group consisting of: H27C/F437C; H27C/T439C; H27C/P440C; H27C/I443C; H27Y; V28M; V30I; N33C/K295C; G37C/S337C; S41C/P283C; Y48C/I169C; Y48C/I169C/A140Y; L49C/L278C; L49F; L49W; I50C/A171C; I50C/T277C; I50W; S52C/K173C; S52C/S275C; S52L; L53C/S174C; P55C/V175C; P55C/Q176C; K56C/N155C; I57F; E58C/I183C; G64C/G196C; G64C/G200C; Q67C/L199C; Q67C/G200C; Y71C/L203C; L86C/V266C; Q89C/A131C; K90Y; I93C/G116C; V94C/G116C; T95C/G116C; T117P; I118C/G381C; A119C/G381C; L120C/G381C; A123P; T124P; S125C/P374C; S125P; S125W; A126P; I128F; I128W; L134C/I267C; A137C/I267C; I144W; L147C/A171C; I151C/A171C; A157C/Q176C; A157C/D177C; A157F; V158L; Q159C/A171C; L168Q; V170I; V170M; A171V; K173Q; V175L; V175P; V179L; E182F; E182W; P185C/A195C; G191P; G200E; I201F; I201W; A202T; E209W; I213C/I226C; I213C/G230C; G219C/E333C; L228C/V264C; L228F; L228W; R236W; R236Y; S246V; L256Y; V264F; V264W; V266F; V266W; S275F; S275M; T277F; T277L; T277W; L278F; L278W; V280F; V280W; R281Y; L282F; L282W; D327C/P344C; A334S; G345M; F346C/T369C; F346C/S370C; N349P; L356F; S361C/T444C; Q362C/N447C; P364C/N447C; T366C/V449C; T367R; N380C/G433C; G381C/K431C; G382C/G433C; V384I; T413C/A436C; G433F; I443W; I443Y; V449C/I454C; V449C/D455C; V449C/I456C; V449C/S457C; A450F; L451P; D452P; I454F; D455K; I456W; S457C/V449C; S457C/I456C; K464C/V449C; S470C/K471C; K471A; K471L; W473A, and wherein the position are relative to SEQ ID NO: 1 or 2.

2. The engineered protein of claim 1, comprising at least one set of paired cysteine substitutions selected from the group consisting of: H27C/F437C; H27C/T439C; H27C/P440C; H27C/I443C; N33C/K295C; G37C/S337C; S41C/P283C; Y48C/I169C; Y48C/I169C/A140Y; L49C/L278C; I50C/A171C; I50C/T277C; S52C/K173C; S52C/S275C; L53C/S174C; P55C/V175C; P55C/Q176C; K56C/N155C; E58C/I183C; G64C/G196C; G64C/G200C; Q67C/L199C; Q67C/G200C; Y71C/L203C; L86C/V266C; Q89C/A131C; I93C/G116C; V94C/G116C; T95C/G116C; I118C/G381C; A119C/G381C; L120C/G381C; S125C/P374C; L134C/I267C; A137C/I267C; L147C/A171C; I151C/A171C; A157C/Q176C; A157C/D177C; Q159C/A171C; P185C/A195C; I213C/I226C; I213C/G230C; G219C/E333C; L228C/V264C; D327C/P344C; F346C/T369C; F346C/S370C; S361C/T444C; Q362C/N447C; P364C/N447C; T366C/V449C; N380C/G433C; G381C/K431C; G382C/G433C; T413C/A436C; V449C/I454C; V449C/D455C; V449C/I456C; V449C/S457C; S457C/V449C; S457C/I456C; K464C/V449C; and S470C/K471C.

3. The engineered protein of claim 1, further comprising at least one set of paired cysteine substitutions selected from the group consisting of: S186C/A195C.

4. (canceled)

5. The engineered protein of claim 1, comprising at least one cavity filling substitution or set of cavity filling substitutions selected from the group consisting of: H27Y; V28M; V30I; L49F; L49W; I50W; S52L; I57F; K90Y; A140Y; I144W; A157F; V158L; V170I; V170M; A171V; V175L; V179L; E182F; E182W; G200E; I201F; I201W; L228F; L228W; R236Y; S246V; L256Y; V264F; V264W; V266F; V266W; S275F; S275M; T277F; T277L; T277W; L278F; L278W; V280F; V280W; R281Y; L282F; L282W; G345M; L356F; V384I; G433F; I443W; I443Y; A450F; I454F; and I456W.

6. (canceled)

7. The engineered protein of claim 1, comprising at least one substitution or set of substitutions selected from the group consisting of: T117P; A123P; T124P; S125P; A126P; V175P; G191P; N349P; L451P; and D452P.

8. The engineered protein of claim 1, comprising at least one substitution or set of substitutions selected from the group consisting of: S125W; I128F; I128W; E209W; and R236W.

9. The engineered protein of claim 1, comprising at least one substitution selected from the group consisting of: K173Q; A202T; A334S; T367R; D455K; K471A; K471L; and W473A.

10. The engineered protein of claim 1, comprising an E at position 108.

11-12. (canceled)

13. The engineered protein of claim 1, comprising a set of substitutions selected from the group consisting of: G64C/G196C/V28M; G64C/G196C/V175L; G64C/G196C/V158L; G64C/G196C/A123P; G64C/G196C/S125P; G64C/G196C/I201W; G64C/G196C/L282F; G64C/G196C/L228W; G64C/G196C/R281Y; G64C/G196C/L282W; G64C/G196C/N349P; G64C/G196C/T367R; G64C/G196C/K471A; A137C/I267C/V28M; A137C/I267C/V175L; A137C/I267C/V158L; A137C/I267C/A123P; A137C/I267C/S125P; A137C/I267C/I201W; A137C/I267C/L282F; A137C/I267C/L228W; A137C/I267C/R281Y; A137C/I267C/L282W; A137C/I267C/N349P; A137C/I267C/T367R; A137C/I267C/K471A; L147C/A171C/V28M; L147C/A171C/V175L; L147C/A171C/V158L; L147C/A171C/A123P; L147C/A171C/S125P; L147C/A171C/I201W; L147C/A171C/L282F; L147C/A171C/L228W; L147C/A171C/R281Y; L147C/A171C/L282W; L147C/A171C/N349P; L147C/A171C/T367R; L147C/A171C/K471A; G64C/G196C/A137C/I267C/K471A; G64C/G196C/A137C/I267C/V175L; G64C/G196C/A137C/I267C/S125P; G64C/G196C/A137C/I267C/S125P/V175L; G64C/G196C/A137C/I267C/T367R; G64C/G196C/A137C/I267C/K471A/S125P; G64C/G196C/A137C/I267C/K471A/S125P/T367R/V175L; G64C/G196C/A137C/I267C; G64C/G196C/A137C/I267C/K471A/S125P/L282F/V175L; G64C/G196C/L147C/A171C/K471A/S125P/L282F/V175L; G64C/G196C/L147C/A171C/A137C/I267C/K471A/S125P/L282F/V175L; G64C/G196C/L147C/A171C; G64C/G196C/L147C/A171C/V28M/V175L/I201W/L228W/S125P/T367R/K471A; G64C/G196C/A137C/I267C/V28M/V175L/I201W/L228W/S125P/T367R/K471A; G64C/G196C/L147C/A171C/A137C/I267C/V28M/V175L/I201W/L228W/S125P/T367R/K471 A; G64C/G196C/I151C/A171C/A137C/I267C; G64C/G196C/I151C/A171C/A137C/I267C/I231C/G230C; G64C/G196C/A137C/I267C/V28M/V175L/I201W/L228W/R281Y; G64C/G196C/A137C/I267C/V28M/V175L/I201W/L282F/L228W/R281Y; I151C/A171C/V449C/S457C; L168Q/G64C/G196C/A137C/I267C/K471A; L168Q/G64C/G196C/A137C/I267C/V175L; L168Q/G64C/G196C/A137C/I267C/S125P; L168Q/G64C/G196C/A137C/I267C/S125P/V175L; L168Q/G64C/G196C/A137C/I267C/T367R; L168Q/G64C/G196C/A137C/I267C/K471A/S125P; L168Q/G64C/G196C/A137C/I267C/K471A/S125P/T367R/V175L; L168Q/G64C/G196C/A137C/I267C; L168Q/G64C/G196C/A137C/I267C/K471A/S125P/L282F/V175L; L168Q/G64C/G196C/I151C/A171C/A137C/I267C; L168Q/G64C/G196C/I151C/A171C/A137C/I267C/I231C/G230C; L168Q/G64C/G196C/A137C/I267C/V28M/V175L/I201W/L228W/R281Y; L168Q/G64C/G196C/A137C/I267C/V28M/V175L/I201W/L282F/L228W/R281Y; L168Q/I151C/A171C/V449C/S457C; L168Q/I151C/A171C/V449C/S457C/V28M/V175L/R281Y; I151C/A171C/S186C/A195C/V28M/V175L/R281Y; L168Q/I151C/A171C/L228W/L282F/R821Y/V175L; I151C/A171C/L228W/L282F/R821Y/V175L; I151C/A171C/L228W/L282F/R821Y/V175L/G200E/I57F; I151C/A171C/L228W/L282F/R821Y/V175L/G200E/I57F/V449C/S457C; I151C/A171C/G200E/I57F; and I151C/A171C/G200E/I57F/V449C/S457C.

14. (canceled)

15. The engineered protein of claim 1, comprising the L168Q substitution.

16-18. (canceled)

19. The engineered protein of claim 1, wherein the protein is fused or conjugated to a trimerization domain.

20. (canceled)

21. The engineered protein of claim 19, wherein the trimerization domain comprises a T4 fibritin trimerization domain, a GCN4 domain, a 4J4A domain, or a combination thereof.

22. (canceled)

23. The engineered protein of claim 1, wherein the protein is fused or conjugated to a transmembrane domain.

24. (canceled)

25. The engineered protein of claim 23, wherein the transmembrane domain comprises a PIV F protein transmembrane domain.

26. (canceled)

27. The engineered protein of claim 23, wherein the transmembrane domain does not comprise a PIV F protein transmembrane domain.

28-30. (canceled)

31. An engineered parainfluenza virus fusion protein (PIV F) trimer comprising three engineered proteins according to claim 1.

32-34. (canceled)

35. A nucleic acid molecule comprising a nucleotide sequence that encodes an amino acid sequence of the engineered protein of claim 1.

36-40. (canceled)

41. A pharmaceutical composition comprising the engineered protein of claim 1; and a pharmaceutically acceptable carrier.

42-44. (canceled)

45. A method of (i) preventing parainfluenza virus (PIV) infection or a disease associated with PIV infection; (ii) eliciting an immune response; or (iii) reducing parainfluenza virus (PIV) viral shedding in a subject, comprising administering to the subject an effective amount of the pharmaceutical composition of claim 41.

46-51. (canceled)

52. A composition comprising the engineered protein of claim 1 bound to an antibody.

53. (canceled)
